(19) United States
(12) Patent Application Publication
(10) Pub. No.: US 2022/0362199 A1
(43) Pub. Date: Nov. 17, 2022

(54) ENRICHED POLYUNSATURATED FATTY ACID COMPOSITIONS

(71) Applicant: NUSEED NUTRITIONAL US INC., West Sacramento, CA (US)

(72) Inventor: Stuart Littler, Laverton (AU)

(73) Assignee: NUSEED NUTRITIONAL US INC., West Sacremento, CA (US)

(21) Appl. No.: 17/771,173

(22) PCT Filed: Oct. 23, 2020
(86) PCT No.: PCT/GB2020/052687

§ 371 Date: Apr. 22, 2022

Related U.S. Application Data
(60) Provisional application No. 62/926,239, filed on Oct. 25, 2019.

(51) Int. Cl. A61K 31/231; A61K 31/201; A61K 31/202

(52) U.S. Cl. CPC A61K 31/231; A61K 31/201; A61K 31/202

(57) ABSTRACT

The present embodiments provide a plant-based lipid composition comprising high concentrations of long-chain omega-3 fatty acids, typically as fatty acid esters, comprising DPA, DTA, ETA, or ETrA, optionally with OA or ALA. These enriched lipid compositions have improved stability, and offer a sustainable source for these long-chain omega-3 fatty acids. In some embodiments, these enriched lipid compositions show enhanced modulation of inflammatory markers, such as inhibition of inflammatory cytokines, and offer nutritional or therapeutic value.

### RELATED APPLICATIONS

This Application is a National Stage Application filed under 35 U.S.C. § 371 of International Application No. PCT/GB2020/052687 filed 23 Oct. 2020, which claims priority benefit of U.S. Provisional Patent Application No. 62/926,239 filed 25 Oct. 2019, the entire contents of which are incorporated fully by reference herein for all purposes.

### FIELD

The present embodiments relate to lipid compositions that are enriched with one or more polyunsaturated fatty acids, for example, omega-3 DPA, DTA, ETA, or combinations thereof. These polyunsaturated fatty acid compositions have a number of health benefits, and enhanced stability. These polyunsaturated fatty acid compositions are obtainable from a single source that is both scalable and sustainable. In some embodiments, omega-3 DPA, DTA, or ETA are combined with oleic acid which synergizes the beneficial activities of these fatty acids.

### BACKGROUND

Long chain omega-3 polyunsaturated fatty acids (LC-omega-3s) are widely recognized as important compounds for human and animal health. These fatty acids may be obtained from dietary sources or to a lesser extent by conversion of linoleic (LA, 18:2 ω-6) or α-linolenic (ALA, 18:3 ω-3) fatty acids, which are regarded as essential fatty acids in the human diet. From a nutritional standpoint, the most important omega-3 fatty acids are probably α-linolenic acid, eicosapentaenoic acid (EPA, 20:5 ω-3 or 20:5n-3), and docosahexaenoic acid (DHA, 22:6 ω-3 or 22:6n-3). For example, DHA is important for brain and eye development; EPA is associated with cardiovascular health.

Docosapentaenoic acid n-3 (DPAn-3, 22:5 ω-3) is a LC-omega-3 with known health benefits, including reducing inflammation and supporting cardiovascular health. DPAn-3 is also a component of adipose, heart, and muscle tissues. Additionally, DPAn-3 is a substrate for conversion to DHA. Accordingly, there is a need for a sustainable source of DPAn-3.

Docosatetraenoic acid (Docosatetraenoic acid, DTAn-3, 22:4 ω-3) is a lesser-known LC-omega-3 with benefits comparatively undocumented. Hence, there remains a need for a sustainable source of DTAn-3, at least to provide for further characterization of this fatty acid.

Eicosatetraenoic acid (ETA, 20:4 ω-3) is a LC-omega-3 that also appears to have anti-inflammatory activities. Additionally, ETA is an intermediate in the biosynthesis of EPA. As with DPA and DTA, there remains a need for a sustainable source of ETA.

Eicosatrienoic acid (ETrA) (C20:3 ω-3) may also serve as an intermediate in the biosynthesis of EPA, as a substrate for conversion to ETA. ETrA may also be implicated in cognitive health. Accordingly, there is a need for a sustainable source of ETrA.

Generally, the oxidative stability of a fatty acid decreases as the number of carbon-carbon double bonds (i.e., the degree of unsaturation) increases. As a consequence, products with increased omega-3 content tend to suffer to reduced shelf life. DPAn-3, DTAn-3 and ETA are all polyunsaturated fats that tend to oxidize readily. Hence, there remains a need for DPAn-3, DTAn-3, or ETA that are stable during or after processing.

### SUMMARY

The present embodiments provide lipid compositions enriched in LC-omega-3 content, such as DPAn-3 (22:5n-3), DTAn-3 (22:4n-3), ETA (20:4n-3), or ETrA (20:3n-3) content, and methods for obtaining these compositions. In at least one embodiment, the DPAn-3, DTAn-3, ETA, or ETrA fatty acid is sourced from a plant, such as plant seed oil from the plant family Brassicaceae. In a particular embodiment, the Brassicaceae is Brassica juncea. In some embodiments, the composition comprises at least one enriched LC-omega-3 (e.g., DPAn-3, DTAn-3, ETA, or ETrA) obtained from a plant source and at least one other LC-omega-3 obtained from another source. A LC-omega-3 of the present embodiments may be in the form of a free fatty acid, a salt, an ester, a salt of an ester, or a combination of these. In at least one embodiment, the LC-omega-3 is in the form of an ethyl ester. In at least one embodiment, the LC-moega-3 is in the form of a triglyceride.

In one aspect, the present embodiments provide for compositions with enriched content of DPAn-3, DTAn-3, ETA, or ETrA, or combinations thereof. For example, at least one embodiment provides a composition comprising about 90%-99% DPAn-3 (inclusive), e.g. about 95% DPAn-3, about 97% DPAn-3, about 98% DPAn-3, or about 99% DPAn-3. Compositions such as these, i.e. compositions which contain very high amounts of DPAn-3 may also comprise a small amount (e.g. at least about 0.1% and up to 5%, up to 2% or up to 1% of oleic acid (OA 18:1n-9). For example, these compositions may comprise about 96% DPAn-3 and about 1% OA. Another embodiment provides a composition comprising about 80%-99% DTAn-3 (inclusive) (optionally together with up to about 15% OA) or about 90%-99% DTAn-3 (inclusive). For example, the composition may comprise about 80% DTAn-3, about 87% DTAn-3, about 90% DTAn-3, or about 95% DTAn-3. Another embodiment provides a composition comprising about 90%-99% ETA (inclusive), e.g. about 93% ETA about 95% ETA, about 98% ETA or about 99% ETA. Another embodiment provides a composition comprising about 60%-70% DPAn-3 (inclusive) and about 0%-20% ETA (inclusive) (such as about 5%-15% ETA (inclusive)), (in particular about 64% DPAn-3 and about 12% ETA). Yet another embodiment provides a composition comprising about 40%-95% DTAn-3 (inclusive) and 5%-60% ETA (inclusive).

In another aspect, the present embodiments provide compositions comprising DPAn-3, ETA, or DTAn-3, and oleic acid (OA, 18: In-9). In at least one embodiment, for example, the composition comprises about 30-60% DTAn-3 (inclusive) and about 30-60% OA (inclusive) (in particular about 49% DTAn-3 and about 43.3% OA). In another embodiment, the composition comprises about 60-80% DTAn-3 (inclusive), about 10-20% OA (inclusive) and about 1-10% ETA (inclusive) (in particular about 74% DTAn-3, about 14% OA and about 4% ETA). In yet another embodiment, the composition comprises about 80-95% DTAn-3 (inclusive) and about 1-15% OA (inclusive) (in particular about 87% DTAn-3 and about 6.3% OA). In yet another embodiment, the composition comprises about 40%-60% DPAn-3 (inclusive), 20%-40% OA (inclusive), and 2%-20% ETA (inclusive). In yet another embodiment, the composition comprises 20%-50% DPAn-3 (inclusive), 10%-30% OA (inclusive), and 2%-20% ETA (inclusive). For example, the composition may comprise 30%-50% DPAn-3 (inclusive), 10%-30% OA (inclusive), and 2%-20% ETA (inclusive) (in particular, the composition may comprise 36% DPAn-3, 22% OA, and 6% ETA). In another embodiment, the composition comprises about 35.8% DPAn-3, about 22.0% OA, and about 6.1% ETA. In an alternative example, the composition may comprise about 5-20% DPA (inclusive), about 30-60% OA (inclusive), and about 1-10% ETA (inclusive) (in particular the composition may comprise about 10.5% DPA, about 44% OA, and about 4% ETA). In another alternative example, the composition may comprise about 20-40% DPAn-3 (inclusive), about 1-10% DTAn-3 (inclusive), about 1-10% ETA (inclusive), about 10-20% ALA (inclusive), about 1-10% LA (inclusive), and about 20-40% OA (inclusive) (in particular the composition may comprise about 28% DPAn-3, about 5% DTAn-3, about 5% ETA, about 14% ALA, about 6% LA, and about 29% OA). In yet another alternative example, the composition may comprise about 10%-40% DPAn-3 (inclusive), about 20%-60% ETrA (inclusive), and about 0%-30% OA (inclusive) (in particular, the composition may comprise about 37% ETrA and about 16% DPAn-3, or it may comprise about 54% ETrA and about 35% DPAn-3) In a related aspect of these embodiments, the composition (that includes OA and at least one of DPAn-3, DTAn-3, or ETA) has synergistic anti-inflammatory activity.

In another aspect, the present embodiments provide compositions comprising at least one enriched fraction of DPAn-3, ETA, ETrA, or DTAn-3, optionally with OA, wherein the composition is anti-inflammatory. In at least one embodiment, the composition modifies cytokine activity. In at least one embodiment, the composition increases cytokine activity associated with decreased inflammation. In at least one embodiment, the composition suppresses cytokine activity associated with increased inflammation. For example, a composition with synergistic anti-inflammatory activity may comprise DPAn-3 and ETA, such as about 64% DPAn-3 and about 12% ETA. In another example, a composition with synergistic anti-inflammatory activity may comprise DTAn-3 and OA, such as, for example, about 3%-95% DTAn-3 (inclusive) and OA, about 49% DTAn-3 and about 43.3% OA, or about 87% DTAn-3 and 6.3% OA. In additional embodiments, the composition enriched for DPAn-3, DTAn-3, or ETA is also enriched for ALA. For example, the composition enriched for DPAn-3 (e.g. containing at least about 28% DPAn-3) may also contain at least about 14% ALA.

In a further aspect, the present embodiments provide a composition enriched for DPAn-3, DTA, ETA or ETrA from a plant (i.e., vegetable matter) source, wherein the composition is more stable than a similar composition in which DPA, DTA, ETA or ETrA is sourced from fish oil or synthetic manufacture, as evidenced by reduced degradation during storage.

The compositions of the present embodiments can be used in feedstuffs, nutraceuticals, cosmetics and other chemical compositions, and they may be useful as intermediates or active pharmaceutical ingredients (APIs).

### DESCRIPTION OF THE DRAWINGS

FIG. 1 is a graph that illustrates better stability of a plant-derived composition enriched in DPAn-3 via double distillation compared with an enriched reference blend. Y-axis: ppm propanal; x-axis: day (0, 3, 5); 0: trans-esterified, double-distilled retentate obtained from B. juncea; □: trans-esterified, double-distilled reference blend.

FIG. 2 is a graph that illustrates better stability of a plant-derived composition enriched in DPAn-3 via double distillation and chromatography (˜98% DPAn-3) compared with a similarly enriched reference blend. Y-axis: ppm propanal; x-axis: day (0, 3, 5); □: trans-esterified, double-distilled, chromatographed fractions obtained from B. juncea (˜98% DPAn-3); □: trans-esterified, double-distilled, chromatographed reference blend HO % EPA).

FIG. 3 s a graph that illustrates better stability of a plant-derived composition enriched in DPAn-3 via double distillation and chromatography (˜64% DPAn-3) compared with an enriched reference blend. Y-axis: ppm propanal; x-axis: day (0, 3, 5); 0: trans-esterified, double-distilled, chromatographed fractions obtained from B. juncea (˜64% DPAn-3); □: trans-esterified, double-distilled, chromatographed reference blend (˜58% EPA).

### DETAILED DESCRIPTION

It should be understood that this invention is not limited to the particular methodology, protocols, and reagents, etc., described herein and as such may vary. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims.

All patents and other publications identified are incorporated herein by reference for the purpose of describing and disclosing, for example, the methodologies described in such publications that might be used in connection with the present embodiments, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on information available to the applicants and do not constitute any admission as to the correctness of the dates or contents of these documents.

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list.

All values are approximate as there is some fluctuation in fatty acid composition due to environmental conditions. Values are typically expressed as percent by weight of total fatty acid, or percent weight of the total seed. Accordingly, other than in the operating examples, or where otherwise indicated, all numbers expressing quantities or reaction conditions used herein should be understood as modified in all instances by the term “about” unless stated to the contrary; “about” refers generally to ±1% of the designated value, but may allow for ±5% or ±10% of the designated value as accepted in the relevant context by one of skill in the art.

The amounts of fatty acids in the compositions of the present embodiments can be determined using routine methods known to those skilled in the art. Such methods include gas chromatography (GC) in conjunction with reference standards, e.g., according to the methods disclosed in the examples provided herein. In a particular method, the fatty acids are converted to methyl or ethyl esters before GC analysis. The peak position in the chromatogram may be used to identify each particular fatty acid, and the area under each peak integrated to determine the amount. As used herein, unless stated to the contrary, the percentage of a particular fatty acid in a sample is determined by calculating the area under the curve in the chromatogram for that fatty acid as a percentage of the total area for fatty acids in the chromatogram. Generally, this corresponds to a weight percentage (w/w or wt %). The identity of fatty acids may be confirmed by gas chromatography-mass spectrometry (GC-MS).

LC-omega-3s are known to have health benefits such as neurological function, diabetes mellitus, cardiovascular health, lipid regulation, and as anti-inflammatory agents. Docosapentaenoic acid (22:5 n-3, or DPAn-3) is valued as an intermediate between EPA and DHA, but it confers many benefits on its own and is, in fact, retro-converted from DHA. DPA is found in high concentrations in mother's milk. Mammalian cells, including human cells, metabolize DPAn-3 to an array of products that are members of the specialized proresolving mediators class of PUFA metabolites that promote restoration of normal cellular function following inflammation that occurs after tissue injury. A study looking at the anti-tumorigenic effects of n-3 fatty acids on colorectal cancer found anti-proliferative and pro-apoptotic effects for EPA, DPA, and DHA, with DPA demonstrating the strongest effects in both in vitro and in vivo models. DPA is also associated with decreased risk in heart disease. See Yazdi, Review of the biologic & pharmacologic role of docosapentaenoic acid n-3, 2 F1000Research 256 (2014). Accordingly, DPA is becoming increasingly important as a nutritional and therapeutic supplement. Recitation of DPA or DPA3 herein refers to DPAn-3 unless noted otherwise.

Compared with a growing wealth of information on DHA and EPA, docosatetraenoic acid (22:4 n-3, DTAn-3) is a relatively little-known LC-omega-3. DTAn-3 is the product of the elongation of ETA. See, e.g., Gregory et al., Cloning and functional characterisation of a fatty acyl elongase from southern bluefin tuna (Thunnus maccoyii), 155 Comp. Biochem. Physiol B Biochem Mol Biol. 178 (2010). Likely because of the similarity of its acyl chain tertiary structure with DPA and DHAn-3, DTAn-3 is associated with beneficial mediation of AP metabolism in the brain. Amtul et al., Structural insight into the different effects of omega-3 & omega-6 fatty acids on the production of Aβ peptides and amyloid plaques, 286 J. Biol. Chem. 6100 (2011). Therefore, DTAn-3 may have potential as a nutritional or therapeutic agent. Recitation of DTA or DTA3 herein refers to DTAn-3 unless noted otherwise.

Eicosatetraenoic acid (ETA, 20:4n-3) is known as an omega-3 intermediate in the biosynthesis of EPA, DPA, and DHA. See, e.g., U.S. Pat. No. 7,807,849. The potential anti-inflammatory activity of ETA has been identified in the context of arthritis. Bierer & Bui, Improvement of arthritic signs in dogs fed green-lipped mussel (Perna canaliculus), 132 J. Nutr. 1623 S (2002). ETA, like other LC-Omega-3 s, may have potential as a nutritional or therapeutic agent.

Eicosatrienoic acid (ETrA) (C20:3n-3) is produced by elongation of ALA or omega-3 desaturation of eicosadienoic acid (EDA, 20:2 n-6), and may be further desaturated to form ETA. See, e.g., U.S. Pat. No. 7,807,849. ETrA is not only an important intermediary in omega-3 pathways, it has been identified as one of the LC-Omega-3s important in cognitive function, at least in bees. Arien et al., Omega-3 deficiency impairs honey bee learning, 112 PNAS 15761 (2015).

Lipid compositions containing LC-omega-3s have typically been obtained from marine sources (e.g., fish, crustacea) or algal sources. Recently, plants have been genetically engineered to produce commercially relevant amounts of LC-omega-3s, particularly DHA. See, e.g., WO 2017/219006; WO 2017/218969. When using these sources, the starting organic matter is first processed in order to extract the oil (generally referred to as the “crude” oil) contained therein. In the case of plant seeds, such as DHA canola or DPA juncea seed, for example, the seeds are crushed to release the oil which is then separated from the solid matter by filtration and/or decanting. If higher concentrations of LC-omega-3 are desired than those found in crude oil, enrichment is required. Further, enrichment may be achieved by processing the crude oil to remove unwanted components (e.g., components which deleteriously affect the product's color, odor or stability, or unwanted fatty acids), while maximizing the levels of the desired fatty acid components. Additionally, if the crude oil is lacking in one or more essential components, it is often blended with crude or enriched oils from other sources (e.g., from fish or algae) to obtain the desired composition.

The compositions of the present embodiments may be obtained from a single source. Particular compositions that may be mentioned in this respect are the products described in Tables 4 and 5 in the Examples, as well as corresponding embodiments of the invention as discussed elsewhere herein. In addition, compositions of the present embodiments which are obtained from a single source may be obtained by providing the lipid mixture obtained from a single source, separating that mixture into a plurality of parts (e.g. via chromatographic separation), and then combining (blending) a subset of those parts. For example, a composition which results from combining two or more fractions obtained in a chromatographic separation method (or other separation method) is contemplated, and those compositions may also be characterised as being obtained from a single source. For example, a composition which contains predominantly DPA and ETA (e.g. wherein the DPA and ETA together make up about 80% by weight of total fatty acid present in the composition) may be obtained from a single source. The combination containing predominantly DPA and ETA may be obtained by blending fractions CXZ and CR in Table 5 in appropriate proportions. Other combinations which are shown to have good activity in vitro may similarly be obtained by blending fractions which are rich in the desired components (e.g. which contain at least 80% of one of said component). The use of a single source facilitates efficient and economic processing of the crude oil and manufacture of the lipid compositions of the invention. “Obtained from a single source” means that the lipid composition is obtainable from one or more organisms of a single taxonomic class. In a particular embodiment, the lipid composition is not derived from multiple organisms across different taxonomic classes and is not, for example, a blend of oils obtained from a combination of fish and algae, or a combination of fish and plants. In this embodiment, the lipid compositions (or the “crude” oils from which the compositions can be obtained by enrichment techniques, such as transesterification, distillation, or chromatography) are obtainable from a single population of organisms, for example, a single source of plant matter or vegetation. In the context of the present embodiment, “vegetable” relates to plants or plant life, as distinct from animal or mineral substances. In other embodiments, however, the compositions are obtained from one plant source (e.g., DPA juncea) and another source (e.g., fish, algae, or synthetic sources).

In at least one embodiment, the lipid composition has a high level of DPAn-3 relative to the amount of other lipids in the composition. In at least one embodiment, the lipid composition has a high level of DTAn-3 relative to the amount of other lipids in the composition. In at least one embodiment, the lipid composition has a high level of ETA relative to the amount of other lipids in the composition. Each of DPAn-3, DTAn-3, or ETA may be independently provided in the form of a free fatty acid, a salt, an ester, or a salt of an ester, or a combination of these, e.g. the composition may contain DPAn-3 in the form of an ester together with ETA in the form of a free fatty acid. In at least one embodiment, the DPAn-3, DTAn-3, or ETA is a fatty acid ester, such as an ethyl ester. These embodiments may contain additional lipid components, such as other omega-3s, saturated, mono- or polyunsaturated fatty acids, in the form of a free fatty acid, a salt, an ester, or a salt of an ester, or a combination of these.

Suitable fatty acid esters forms are known to the skilled person. For example, fatty acid ester forms that are nutritionally acceptable or pharmaceutically acceptable include ethyl esters, methyl esters, phospholipids, monoglycerides, diglycerides and triglycerides (triacylglycerides) of fatty acids. Different ester forms may be required depending on the intended use of the lipid composition. For example, triglycerides are esters derived from glycerol and three fatty acids. Triglycerides are particularly suited for use in foods intended for human consumption, especially infant consumption, due in part to the taste and the stability of these ester forms to heat treatment (which may be necessary for such food products). Accordingly, one embodiment provides a food product for human or animal consumption comprising a lipid composition of the invention, wherein the DPAn-3, DTAn-3, or ETA polyunsaturated fatty acids are provided in the form of triglyceride esters. Ethyl esters are particularly suited for use in dietary supplements as these ester forms can be manufactured efficiently and easily. Thus, in at least one embodiment, the DPAn-3, DTAn-3, or ETA is independently provided in the form of a fatty acid ethyl ester.

Alternatively, fatty acid components may alternatively be present in the form of “free” fatty acids, i.e., the —COOH form of the fatty acid. In particular compositions of the invention, the compositions contain relatively low levels of fatty acids in this form because they are associated with an unpleasant (often “soapy”) taste, and are less stable than esterified fatty acids. Free fatty acids are typically removed from lipid compositions by way of alkali or physical refining as are well-known in the art. Accordingly, in one embodiment the total free fatty acid content in the lipid compositions is less than 5% (such as less than 3%, particularly less than 2%) by weight of the total fatty acid content of the composition.

The lipid compositions of the present embodiments may also contain other components (e.g., other than fatty acids noted above) that originate from the source material and that are not fully removed during extraction and enrichment processes. The precise identities of those other components may vary greatly depending on the source material, but examples of such other components include phytosterols (i.e., plant sterols and plant stanols) present either as a free sterol or as a sterol ester (such as β-sitosterol, β-sitostanol, Δ5-avenasterol, campesterol, Δ5-stigmasterol, Δ7-stigmasterol and Δ7-avenasterol, cholesterol, brassicasterol, chalinasterol, campesterol, campestanol, or eburicol). Other example components include antioxidants, such as tocopherols and tocotrienols. Thus, particular lipid compositions of the present embodiments may include those which contain detectable quantities of one or more phytosterols (such as β-sitosterol). Such sterols may be present at about 0.01% or more, but typically not more than about 1% (e.g., wt %) of the lipid composition.

The compositions of the present embodiments are advantageously obtainable from plant sources (“vegetable” sources). “Vegetable-based” refers to at least 70% by weight of the lipids that are present in the lipid compositions are obtained from vegetable sources. Vegetable sources include plant sources, particularly crops such as oil seed crops. In at least one embodiment, lipids are obtained from a seed oil crop such as Brassica, for example B. juncea or B. napus. For the avoidance of doubt, however, it is not essential that the compositions be obtained solely from such sources: a proportion (e.g., at most about 30% by weight) of the lipids in the compositions of the embodiments may be obtained from other sources, including marine oils (e.g., from fish or crustacea), algal oils, or mixtures thereof. In one example at least 80%, such as at least 90%, by weight of the lipids that are present are obtained from vegetable sources. In particular embodiments, essentially all (i.e. at least 95%, at least 99%, or about 100%) of the lipids are obtained from vegetable sources.

The use of plants as a lipid or fatty acid source offers a number of advantages. For example, marine sources of oils are known to contain relatively high levels of contaminants (such as mercury, PCBs, or fish allergens (e.g., parvalbumins)) that are not found in plant materials. Further, historic overfishing has also depleted the stocks of fish and crustacea (e.g., krill) such that they are no longer sustainable. The present invention therefore offers a sustainably-sourced polyunsaturated fatty acid oil composition containing relatively low levels of unwanted contaminants.

Accordingly, the present lipid compositions (and the feedstuffs and pharmaceutical compositions comprising these compositions) are not of animal (e.g., marine animal) origin. That is, in such embodiments the lipid compositions do not contain any components that are sourced from animals, such as fish and crustacea. Lipid compositions in which no components are obtained from an animal are believed to be advantageous in terms of lipid content and a stability profile that can be achieved following standard refinement or enrichment procedures.

In an aspect of the embodiments, the lipid composition is derived from a plant. Plants from which the oils are obtained are typically oilseed crops, such as mustard, canola, copra, cottonseed, flax, palm kernel, peanut, rapeseed, soybean, and sunflower seed. Compositions obtained exclusively from plants may be referred to as “vegetable” oils or “vegetable lipid compositions.” Suitable plants from which the lipid compositions of the embodiments may be obtained (whether or not at commercial scale) are known to the skilled person and include Brassica sp. (oilseed such as B. juncea, B. napus, or B. carinata), Arabidopsis thaliana (cress), Linum usitatissimum (flax), Camelina sativa (false flax), Gossypium hirsutum (cotton), Helianthus sp. (sunflower), Carthamus tinctorius (safflower), Glycine max (soybean), Zea mays (corn), Sorghum sp., Avena sativa (oats), Trifolium sp. (clover), Nicotiana sp. (e.g., benthamiana or tabacum) tobacco), Hordeum vulgare (barley), Lupinus angustifolius (lupine), Oryza sp. (rice such as O. sativa or O. glaberrima), Elaesis guineenis (palm), or Crambe abyssinica (crambe, an oilseed of Brassicaceae). In at least one embodiment, the plant source is Brassica sp.

Suitable sources (including marine and algal sources in addition to plant sources) may be naturally occurring, or may be genetically modified for the ability to produce omega-3s. Examples of plant sources that have been genetically modified for this purpose are known to the skilled person. See, e.g., WO 2013/185184, WO 2015/089587, WO 2015/196250. For example, genetically engineered canola line NSB500274, that produces DHA in its seed oil, is described in WO 2017/218969 and WO 2017/219006. Processes for obtaining oils from suitable sources are well-known in the art. Enrichment of the described omega-3s from these oils is discussed herein.

Techniques that are routinely practiced in the art can be used to extract, process, and analyze oils produced by plants and seeds. Briefly plant seeds are typically cooked, pressed, and oil extracted to produce crude oil. That oil may, in turn, be degummed, refined, bleached, or deodorized. A combination of degumming, refining, bleaching and deodorizing has been found to be particularly effective for preparing LC-omega-3-enriched lipid mixtures. Thus, in one embodiment, the lipid composition is obtained from a seed oil that has been degummed, refined, bleached and/or deodorized. It is not always necessary for the oils to be processed in this way, however, and adequate purification and enrichment may be achieved without these methods.

Generally, techniques for crushing seed are known in the art. For example, oilseeds can be tempered by spraying them with water to raise the moisture content to, e.g., 8.5%, and flaked using a smooth roller with a gap setting of 0.23 mm to 0.27 mm. Depending on the type of seed, water may not be added prior to crushing. Extraction may also be achieved using an extrusion process. The extrusion process may or may not be used in place of flaking, and is sometimes used as an add-on process either before or after screw pressing.

In an embodiment, the majority of the seed oil is released by crushing using a screw press. Solid material expelled from the screw press is then extracted with a solvent, e.g., hexane, using a heated column, after which solvent is removed from the extracted oil. Alternatively, crude oil produced by the pressing operation can be passed through a settling tank with a slotted wire drainage top to remove the solids that are expressed with the oil during the pressing operation. The clarified oil can be passed through a plate and frame filter to remove any remaining fine solid particles. If desired, the oil recovered from the extraction process can be combined with the clarified oil to produce a blended crude oil. Once the solvent is stripped from the crude oil, the pressed and extracted portions are combined and subjected to normal oil processing procedures.

As used herein, “purified,” when used in connection with lipid or oil described herein typically means that that the extracted lipid or oil has been subjected to one or more processing steps to increase the purity of the lipid/oil component. For example, purification steps may comprise one or more of: degumming, deodorizing, decolorizing, or drying the extracted oil. The term “purified” does not, however, include a transesterification process or another process that alters the fatty acid composition of the lipid or oil of the invention so as to increase the LC-omega-3 content as a percentage of the total fatty acid content. In other words, the fatty acid compositions comprising the purified lipid or oil is essentially the same as that of the unpurified lipid or oil.

Plant oils may be refined (purified) once extracted from the plant source, using one or more of the following process, and particularly using a combination of degumming, alkali refining, bleaching and deodorization. Suitable methods are known to those skilled in the art. See, e.g., WO 2013/185184.

Briefly, degumming is an early step in the refining of oils and its primary purpose is the removal of most of the phospholipids from the oil. Addition of about 2% water, typically containing phosphoric acid, at 70° C. to 80° C., to the crude oil results in the separation of most of the phospholipids accompanied by trace metals and pigments. The insoluble material that is removed is mainly a mixture of phospholipids. Degumming can be performed by addition of concentrated phosphoric acid to the crude seed oil to convert nonhydratable phosphatides to a hydratable form, and to chelate minor metals that are present. Typically, gum is separated from the seed oil by centrifugation.

Alkali refining is one of the refining processes for treating crude oil, sometimes also referred to as neutralization. It usually follows degumming and precedes bleaching. Following degumming, the seed oil can be treated by the addition of a sufficient amount of an alkali solution to titrate all of the free fatty acids and phosphoric acid, and removing the soaps thus formed. Suitable alkaline materials include sodium hydroxide, potassium hydroxide, sodium carbonate, lithium hydroxide, calcium hydroxide, calcium carbonate and ammonium hydroxide. Alkali refining is typically carried out at room temperature and removes the free fatty acid fraction. Soap is removed by centrifugation or by extraction into a solvent for the soap, and the neutralized oil is washed with water. If required, any excess alkali in the oil may be neutralized with a suitable acid such as hydrochloric acid or sulfuric acid.

Bleaching is a refining process in which oils are heated at 90° C. to 120° C. for 10 to 30 minutes in the presence of a bleaching earth (0.2% to 2.0%) and in the absence of oxygen by operating with nitrogen or steam or in a vacuum. Bleaching is designed to remove unwanted pigments (carotenoids, chlorophyll, etc.), and the process also removes oxidation products, trace metals, sulfur compounds and traces of soap.

Deodorization is a treatment of oils and fats at a high temperature (e.g., about 180° C.) and low pressure (0.1 mm Hg to 1 mm Hg). This is typically achieved by introducing steam into the seed oil at a rate of about 0.1 ml/minute/100 ml of seed oil. After about 30 minutes of sparging, the seed oil is allowed to cool under vacuum. This treatment improves the color of the seed oil and removes a majority of the volatile substances or odorous compounds, including any remaining free fatty acids, monoacylglycerols, and oxidation products.

Winterization is a process sometimes used in commercial production of oils for the separation of oils and fats into solid (stearin) and liquid (olein) fractions by crystallization at sub-ambient temperatures. It is typically used to decrease the saturated fatty acid content of oils.

The present embodiments relate, in part, to lipid compositions obtained using transesterification techniques. As noted herein, crude oils usually contain the desired fatty acids in the form of triacylglycerols (TAGs). Transesterification is a process that can be used to exchange the fatty acids within and between TAGs or to transfer the fatty acids to another alcohol to form an ester (such as an ethyl ester or a methyl ester). In embodiments of the described herein, transesterification is achieved using enzymatic or chemical means.

Regarding enzymatic transesterification, in this approach transesterification is achieved using one or more enzymes, particularly lipases that are known to be useful for hydrolyzing ester bonds, e.g., in glycerides. The enzyme may be a lipase that is position-specific (sn-1/3 or sn-2 specific) for the fatty acid on the triacylglyceride (triglyceride or TAG), or that has a preference for some fatty acids over others. Particular enzymes that may be mentioned include Lipozyme 435 (available from Novozymes). The process is typically performed at ambient temperature. The process is typically performed in the presence of an excess quantity of the alcohol corresponding to the desired ester form (e.g., by using ethanol in order to form ethyl esters of the fatty acids).

Chemical transesterification uses a strong acid or base as a catalyst. Sodium ethoxide (in ethanol) is an example of a strong base that is used to form fatty acid ethyl esters through transesterification. The process may be performed at ambient temperature or at elevated temperature (e.g., up to about 80° C.).

In at least one embodiment, the enriched lipid composition of the present embodiments is obtained using distillation. Molecular distillation is an effective method for removing significant quantities of the more volatile components, such as short-chain saturated fatty acids, from crude oils. Distillation is typically performed under reduced pressure, e.g., below about 1 mbar. The temperature and duration of the process may then be chosen to achieve an approximately 50:50 split between the distillate and residue after a distillation time of a few hours (e.g., 1 hour to 10 hours). Typical distillation temperatures used in the production of the lipid compositions of the present invention are in the region of 120° C. to 180° C., such as between 140° C. and 160° C., particularly between 145° C. and 160° C.

Multiple distillations may be performed, with each distillation being deemed complete when an approximately 50:50 split between the distillate and residue is achieved. The use of successive distillations reduces the overall yield, but two distillations (i.e., the product is referred to as “double distilled”) may produce optimal results.

In addition to distillation, chromatography is an effective method for separating the various components of fatty acid mixtures. Chromatography may be used to increase the concentration of one or more preferred LC-omega-3s within a mixture. Chromatographic separation can be achieved under a variety of conditions, but it typically involves using a stationary bed chromatographic system or a simulated moving bed system. These are explained as follows.

A stationary bed chromatographic system is based on the following concept: a mixture whose components are to be separated is (normally together with an eluent) caused to percolate through a column containing a packing of a porous material (the stationary phase) exhibiting a high permeability to fluids. The percolation velocity of each component of the mixture depends on the physical properties of that component so that the components exit from the column successively and selectively. Thus, some of the components tend to fix strongly to the stationary phase and thus will be more delayed, whereas others tend to fix weakly and exit from the column after a short while.

A simulated moving bed system consists of a number of individual columns containing adsorbent which are connected together in series and which are operated by periodically shifting the mixture and eluent injection points and also the separated component collection points in the system whereby the overall effect is to simulate the operation of a single column containing a moving bed of the solid adsorbent. Thus, a simulated moving bed system consists of columns which, as in a conventional stationary bed system, contain stationary beds of solid adsorbent through which eluent is passed, but in a simulated moving bed system the operation is such as to simulate a continuous countercurrent moving bed.

Columns used in these processes typically contain silica (or a modified silica) as the basis for the stationary phase. The mobile phase (eluent) is typically a highly polar solvent mixture, often containing one or more protic solvents, such as water, methanol, ethanol, and the like, as well as mixtures thereof. The eluent flow rate may be adjusted by the skilled person to optimize the efficiency of the separation process. For example, the products defined in the claims may be obtained using a relatively fast eluent flow rate. The use of slower flow rates may improve the degree of separation of the FAs contained in the initial mixture thus enabling higher concentrations or purer fractions of DPA to be obtained. Detection methods for LC-PUFAs are known to those skilled in the art, and include UV-vis absorption methods as well as refractive index detection methods.

Accordingly, another aspect of the present embodiments provides a process for producing a lipid composition, wherein the process comprises providing a mixture of fatty acid ethyl esters, then subjecting the mixture to a chromatographic separation process. The present embodiments also provide lipid compositions that are obtainable by such processes. Suitable chromatographic separation conditions include those described herein.

For example, preparative high performance liquid chromatography (HPLC) techniques can be used to obtain enriched lipid fractions. A particular mobile phase that may be used in the chromatographic separation is a mixture of methanol and water (e.g., 88% methanol), though this may be changed (e.g., to increase the methanol content) during the separation process to enhance the efficiency. A particular stationary phase that may be used is a silica-based stationary phase. Analytical HPLC, or any other suitable technique known to the person skilled in the art, may be performed on the fractions obtained to identify those that contain sufficiently high concentrations of the desired fatty acids, and thus contain the lipid compositions of the invention.

Accordingly, in at least one embodiment, enriched fatty acid ethyl esters are obtained by transesterification and distillation of a vegetable-based lipid oil, e.g., via any one of the processes hereinbefore described. The vegetable-based lipid oil may be obtained from any of the plants, particularly the oilseeds, disclosed herein or otherwise known in the art. Prior to transesterification and distillation, refinement of the vegetable-based lipid oil using degumming, alkali refinement, bleaching or deodorization may optionally be performed.

The lipid compositions of the present invention are useful as active pharmaceutical ingredients (APIs) or as precursors (or intermediates) to APIs that may be obtained therefrom by way of further enrichment. Such compositions would be further enriched in the levels of beneficial LC-omega-3s, such as DPAn-3, DTAn-3, ETA, or combinations thereof, or mixtures of the preceding with OA or ALA. The form of these LC-omega-3s may be any pharmaceutically acceptable form, such as free fatty acid, ethyl ester, triglyceride, or combinations thereof.

The concentration of fatty acids in an oil can be increased further by a variety of methods known in the art, such as, for example, freezing crystallization, complex formation using urea, supercritical fluid extraction and silver ion complexing. Complex formation with urea is a simple and efficient method for reducing the level of saturated and monounsaturated fatty acids in the oil. Initially, the TAGs of the oil are split into their constituent fatty acids, often in the form of fatty acid esters. These free fatty acids or fatty acid esters, which are usually unaltered in fatty acid composition by the treatment, may then be mixed with an ethanolic solution of urea for complex formation. The saturated and monounsaturated fatty acids easily complex with urea and crystallize out on cooling and may subsequently be removed by filtration. The non-urea complexed fraction is thereby enriched with LC-omega-3 fatty acids (although shorter-chain polyunsaturated omega-3 or omega-6 fatty acids may be enriched by this technique).

The lipid compositions of the present embodiments may be bulk oils in which the lipid composition has been separated from the source matter (e.g., plant seeds) from which some or all of the lipid was obtained.

The lipid compositions of the present embodiments can be used in or as feedstuffs. That is, these compositions of the may be provided in an orally available form. For purposes of the present embodiments, “feedstuffs” include any food or preparation for human consumption which when taken into the body serves to nourish or build up tissues or supply energy; and/or maintains, restores or supports adequate nutritional status or metabolic function. Feedstuffs include nutritional compositions for babies or young children such as, for example, infant formula. In the case of feedstuffs, the fatty acids may be provided in the form of triglycerides in order to further minimize any unpleasant tastes and maximize stability.

Feedstuffs comprise a lipid composition as described herein, optionally together with a suitable carrier. The term “carrier” is used in its broadest sense to encompass any component that may or may not have nutritional value. As the skilled person will appreciate, the carrier must be suitable for use (or used in a sufficiently low concentration) in a feedstuff such that it does not have deleterious effect on organisms consuming the feedstuff. The feedstuff composition may be in a solid or liquid form.

Additionally, the composition may include edible macronutrients, protein, carbohydrate, vitamins, or minerals in amounts desired for a particular use as are well-known in the art. The amounts of these ingredients will vary depending on whether the composition is intended for use with normal individuals or for use with individuals having specialized needs, such as individuals suffering from metabolic disorders and the like.

Examples of suitable carriers with nutritional value include macronutrients such as edible fats (e.g., coconut oil, borage oil, fungal oil, black current oil, soy oil, mono- or diglycerides), carbohydrates (e.g., glucose, edible lactose, hydrolyzed starch) and proteins (e.g., soy proteins, electro-dialyzed whey, electro-dialyzed skim milk, milk whey, or the hydrolysates of these proteins).

Vitamins and minerals that may be added to the feedstuff disclosed herein include, for example, calcium, phosphorus, potassium, sodium, chloride, magnesium, manganese, iron, copper, zinc, selenium, iodine, and Vitamins A, E, D, C, and the B complex.

In another aspect of the present embodiments, the lipid compositions can be used in pharmaceutical compositions. Such pharmaceutical compositions comprise the lipid composition of the embodiments optionally together with one or more pharmaceutically acceptable excipients, diluents or carriers, which are known to the skilled person. Suitable excipients, diluents or carriers include phosphate-buffered saline, water, ethanol, polyols, wetting agents or emulsions such as a water/oil emulsion. The composition may be in either a liquid or solid form, including as a solution, suspension, emulsion, oil, or powder. For example, the composition may be in the form of a capsule tablet, encapsulated gel, ingestible liquid (including an oil or solution) or powder, emulsion, or topical ointment or cream. The pharmaceutical composition may also be provided as an intravenous preparation.

Particular forms suitable for feedstuffs and for pharmaceutical compositions include liquid containing capsules and encapsulated gels. The lipid compositions of the invention may be mixed with other lipids or lipid mixtures (particularly vegetable-based fatty acid esters and fatty acid ester mixtures) prior to use. The lipid compositions of the invention may be provided together with one or more additional components selected from the group consisting of an antioxidant (e.g., a tocopherol such as α-tocopherol or γ-tocopherol, or a tocotrienol), a stabilizer, and a surfactant. Tocopherols and tocotrienols are naturally occurring components in various plant seed oils, including canola oils.

It may also be desirable to include isotonic agents, for example, sugars, sodium chloride, and the like. Besides such inert diluents, the composition can also include adjuvants, such as wetting agents, emulsifying and suspending agents, sweetening agents, flavoring agents and perfuming agents. Suspensions, in addition to the lipid compositions of the invention, may comprise suspending agents such as ethoxylated isostearyl alcohols, polyoxyethylene sorbitol and sorbitan esters, microcrystalline cellulose, aluminum metahydroxide, bentonite, agar-agar, and tragacanth or mixtures of these substances.

Solid dosage forms such as tablets and capsules can be prepared using techniques well known in the art. For example, fatty acids produced in accordance with the methods disclosed herein can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with the relevant lipid composition and optionally one or more antioxidants.

Possible routes of administration of the pharmaceutical compositions of the present embodiments include, for example, enteral (e.g., oral and rectal) and parenteral. For example, a liquid preparation may be administered orally or rectally. Additionally, a homogenous mixture can be completely dispersed in water, admixed under sterile conditions with physiologically acceptable diluents, preservatives, buffers or propellants to form a spray or inhalant.

Lipid compositions described herein may provide a number of benefits that are typically associated with long-chain polyunsaturated fatty acids. For example, the lipid compositions and the pharmaceutical compositions described hereinabove, may be used in the treatment or prevention of cardiovascular disease, protection against death in patients with cardiovascular disease, reduction of overall serum cholesterol levels, reduction in high BP (blood pressure), increase in HDL:LDL ratio, reduction of triglycerides, or reduction of apolipoprotein-B levels, as may be determined using tests that are well-known to the skilled person. Accordingly, an aspect of the present embodiments provides methods of treating (or preventing) the diseases and conditions using the lipid compositions described herein.

As used herein, the terms “treatment,” “treat,” and “treating” refer to reversing, alleviating, inhibiting the progress of a disease or disorder as described herein, or delaying, eliminating or reducing the incidence or onset of a disorder or disease as described herein, as compared to that which would occur in the absence of the measure taken. As used herein, the terms “prevent,” “prevention” and “preventing” refer to the reduction in the risk of acquiring or developing a given condition, or the reduction or inhibition of the recurrence or said condition in a subject who is not ill.

A typical dosage of a particular fatty acid is from 0.1 mg to 20 g, taken from one to five times per day (up to 100 g daily), and is particularly in the range of from about 10 mg to about 1 g, 2 g, 5 g, or 10 g daily (taken in one or multiple doses). As known in the art, a minimum of about 300 mg/day of fatty acid, especially LC-omega-3, is desirable. It will be appreciated, however, that any amount of fatty acid will be beneficial to the subject. Oral dosage forms may be taken with a meal to increase absorption of the omega-3 fatty acid(s). When used as a pharmaceutical composition, the dosage of the lipid composition to be administered to the patient may be determined by one of ordinary skill in the art and depends upon various factors such as weight of the patient, age of the patient, overall health of the patient, past history of the patient, immune status of the patient, etc.

The compositions of the present embodiments are readily obtainable compositions that may have an improved stability profile and which may contain a mixture of fatty acids in which the relative proportions of omega-3 and omega-6 fatty acids are particularly beneficial for human health. Stability may be assessed using a variety of methods known to those skilled in the art. Such methods include the Rancimat method, the assessment of propanal formation (particularly appropriate for omega-3 fatty acids), the assessment of hexanal formation (particularly appropriate for omega-6 fatty acids), the “peroxide value” method (e.g., using AOCS official method Cd 8-53) and the “p-anisidine value” method (e.g., using AOCS official method Cd 18-90). It is shown in the Examples that the compositions of the present embodiments have a better stability profile than reference blends (the reference blends having a similar composition in terms of the key LC-PUFAs but containing a significant quantity of lipid of animal (fish) or synthetic origin).

The compositions of the present embodiments may also have the advantage in efficacy, less toxicity, half-life, potency, fewer sequelae, metabolism, or pharmacokinetic profile (e.g., higher oral bioavailability and/or lower clearance) or other useful pharmacological, physical, or chemical properties compared with lipid compositions of the prior art.

### EXAMPLES

### Example 1—DPA Juncea Oil Extraction from Seeds

Brassica juncea NUBJ1207 (deposited ATCC Accession No. PTA-125954) was grown in tents in California, USA. The seed was harvested and then stored at room temperature prior to crushing. NUBJ1207 produces high amounts of DPAn-3 in its seed oil (more than 10%).

Seed (4.92 kg) was crushed to produce DPA oil using a Kern Kraft KK80 screw press. The expeller collar heater temperature was set to the maximum set temperature on the thermostat. Initial ambient and choke temperature was 20° C. and the choke distance was set at 73.92 mm. The seed was fed with continual oil and meal collection without stopping the expeller till all the seed was crushed.

The speed of rotation of the auger, the temperature of the meal and expelled oil were monitored throughout the pressing. A yield of 1.02 kg (20.7%) crude oil was obtained. After filtering to remove fines, the yield was 0.96 kg (19.4%). The oil profile (fatty acid content) of this preparation (designated BrJ) is shown in Table 1.

### Example 2—Reference Blend Oil

Pure fish oil contains low levels of ALA fatty acids and significantly higher levels of EPA and DHA. A reference oil blend was designed to be similar in composition to the filtered DPA Juncea oil obtained in Example 1. Because DPA was not available from other sources in comparable amounts, EPA was selected as a comparator for inclusion in the reference oil because it has five double bonds. This was achieved by blending a fish oil rich in EPA, flaxseed oil, and high OA sunflower oil. The resulting reference blend oil also had a similar total omega-3 content to the DPA Juncea oil.

More specifically, to a dry, nitrogen flushed reactor fitted with a mechanical stirrer was added semi-refined sardine oil (19.40 kg, 48.5%), crude high oleic sunflower oil (9.52 kg, 23.8%) and crude flax seed oil (11.08 kg, 27.7%), and the mixture stirred in an inert atmosphere at ambient temperature for 2 hr. The reference oil (designated Rf) was drained from the reactor and stored under nitrogen prior to use.

TABLE 1
compares example DPA-Juncea and reference blend oils:
Table 1. Major FA in starting oils (%, GC-FID)

| | Crude DPA-Juncea | Crude reference |
|---|---|---|
| Fatty acid (FA) | oil (BrJ) | blend oil (Rf) |
| OA (18:1n9) | 43.73 | 26.71 |
| LA (18:2n6) | 7.64 | 8.60 |
| ALA (18:3n3) | 16.51 | 16.94 |
| EPA (20:5n3) | 0.54 | 10.19 |
| DPA (22:5n3) | 10.46 | 1.0 |
| EPA, DPA, DHA | 11.0 | 14.55 |

### Example 3—Enzymatic Trans-Esterification of Crude DPA Juncea Oil

The following enzymatic trans-esterification procedure was performed on about 5 kg of the crude triglyceride oil obtained in Example 1 to produce fatty acid ethyl ester (FAEE).

To a dry, nitrogen flushed reactor fitted with a mechanical stirrer was added 100% undenatured ethanol (2.0 kg) and the crude triglyceride oil obtained in Example 1 (0.95 kg) and the mixture stirred. To this mixture was added 100 g Lipozyme 435 (Novozymes A/S) and the mixture heated at 40° C. for 21 hr. A ¹H NMR spectrum recorded of a sample taken from the mixture indicated the reaction was complete.

The mixture was cooled to 20° C. The mixture was drained from the reactor and filtered through a 4 μm polypropylene filter cloth on a 20 L Neutsche filter. The reactor was rinsed with ethanol (2×1.25 L) and pet. spirit (2.5 L) and these used to sequentially wash the filter cake. To the resulting crude reaction mixture was added pet. spirit (2.5 L) and water (2 L) and the mixture thoroughly mixed in the reactor and then allowed to stand, after which two phases formed.

The pet. spirit layer was removed and the aqueous layer further extracted with pet. spirit (1×5 L and 1×2.5 L). The combined pet. spirit layers were dried over anhydrous magnesium sulphate (approx. 1 kg), filtered and concentrated in vacuo to give a yellow oil (yield: 99%) crude DPA FAEE. Yield: 99.0%.

Enzymatic transesterification of the crude triglyceride reference blend oil obtained according to Example 2 (5.0 kg) was completed using the process as just described. The product FAEE was obtained as a yellow oil.

### Example 4—Enrichment by Vacuum Distillation of Transesterified Oils

The following describes a standard procedure for the removal of more volatile components of fatty acid ethyl esters (FAEE) mixtures by vacuum distillation. The FAEE from the Example 3 were subjected to distillation to develop two fractions, a distillate fraction containing little DPAEE and a residue fraction containing most of the DPAEE which is less volatile. Separation by distillation was achieved by passing the trans-esterified crude oil through a Pope 2-inch (50 mm) wiped film still under vacuum equipped with 2×1000 ml collection flasks collecting the distillate and residue. Each was analyzed for fatty acid composition. Vacuum was supplied by an Edwards 3 rotary pump and the vacuum measured by an ebro-vacumeter VM2000.

The oil was fed into the still by a Cole-Palmer Instrument Company easy-load II peristaltic pump at 4 mL/min with the still motor set to 325 rpm with water condenser used to condense the distillate. The feed was continued until such time as one or other of the receiver flasks was full (such that relatively equal portions of distillate and retentate oils were observed). Crude DPA FAEE was distilled under these conditions with the heater bands initially set to 153° C. The objective was to obtain a 50:50 split of distillate:residue. During the first 45 minutes of the experiment, the temperature of the heater bands was decreased to 143° C. to decrease the proportion of the oil that distilled and the still then allowed to equilibrate. After minutes, the temperature of the heater bands was adjusted down to 141° C. The remainder of the distillation took place at 141° C. The total time of distillation was 145 min. Yield: 52.1% distillate, 47.1% retentate (residue). The volume was thus reduced by 50% while maintaining 80% of the DPAEE in the residual oil. This produced oils with about 19% DPAEE from oil that originally contained about 10% DPAEE.

A portion of the residue from the above distillation was again subjected to the removal of more volatile components by distillation under the standard conditions with the temperature of the heater bands set to 145° C., again targeting a 50:50 split. Over 20 minutes, in an attempt to increase the proportion of distilled oil, the temperature of the heater bands was increased to 153° C., and held at that temperature. After 50 min at 153° C. however, the flow rate of the distillate was found to be too high and the temperature of the heater bands was reduced to 151° C. for the remainder of the distillation. The total time of distillation was 95 min. Yield: 53.0% distillate, 46.4% retentate. This produced an oil with about 35% DPAEE. The end result of the double distillation reduced the volume of oil be a factor of 4, while retaining about 65% of the original DPAEE in the oil.

The reference oil was distilled under similar conditions.

### Example 5— Chromatographic Separation of DPA-Juncea-Derived FAEE

The FAEE obtained in Example 4 (i.e., FAEE that had been obtained from the crude DPA-Juncea oil by transesterification and double-distillation) were subjected to chromatographic separation via preparative HPLC. Preparative HPLC on a 1-g-scale followed by vacuum concentration produced fractions greater than 85% DPAEE (and greater than 85% EPAEE from reference blend oils). A second preparative HPLC experiment was performed to obtain a single fraction of either 50-85% DPA-enriched oil or 40-60% EPA-enriched oil. Additional preparative HPLC experiments were performed using an alternative column. All other FAEE fractions were collected and analyzed for purity by HPLC and pure fractions of interest were concentrated under vacuum. In this way, fractions enriched in OAEE, LAEE, ALAEE, ETAEE, EPAEE, and DPAEE were also produced from one or more of the oils.

Preparative HPLC method A: This method used an HPLC system comprising a Waters Prep 4000 system, Rheodyne injector with 10 ml loop, 300×40 mm Deltaprep C18 column, Waters 2487 dual wavelength detector and chart recorder was equilibrated with 88% methanol/water mobile phase at 70 mL/min. The detector was set to 215 nm and 2.0 absorbance units full scale and the chart run at 6 cm/hr. 1.0 g of FAEE oil was dissolved in a minimum amount of 88% methanol/water and injected onto the column via the Rheodyne injector. Approximately 250 mL fractions were collected once the solvent front appeared after about 7 min. Forty-seven (47) fractions were collected over 150 min. After 106 min, the mobile phase was changed to 90% methanol/water. After 116 min, the mobile phase was changed to 94% methanol/water. After 134 min, the mobile phase was changed to 100% methanol. After the final fractions were collected, the column was washed for a further 1 hr with 100% methanol at 70 mL/min.

Analytical HPLC was performed on all fractions (and HPLC methods A-D, above and below), and the “symmetrical” fractions containing predominantly DPA were combined (yield: 22%). A HPLC system comprising a Waters 600E pump controller, 717 autosampler, 2996 photodiode array detector and 2414 refractive index detector was used for sample analysis. The analysis was performed on a 150×4.6 mm Alltima C18 column using either isocratic 90% methanol/water or 95% methanol/water at 1.0 mL/min as mobile phase. Data collection and processing was performed in Waters Empower 3 software.

This approach yielded fractions designated “A fr” in Table 2:

TABLE 2
Yield from preparative HPLC method A

| | Mass FAEE | Significant |
|---|---|---|
| Fraction | obtained | component(s)* |
| A fr 18 | 2.5 mg | ALA |
| A fr 19-21 | 57 mg | ALA |
| A fr 23-26 | 40 mg | ETA |
| A fr 27-28 | 23 mg | LA, DPA3 |
| A fr 29-34 | 228 mg | DPA3 |
| A fr 35-37 | 17 mg | OA, ETrA, DPA3 |
| A fr 38 | 108 mg | OA |
| A fr 39-40 | 77 mg | OA, DTA3 |
| A fr 44-45 | 38 mg | |

*At least 10% FAEE.

In a variation of this method for isolating a high-purity DPAEE, method A was modified as follows: After 96 min the mobile phase was changed to 90% methanol/water. After 109 min the mobile phase was changed to 94% methanol/water. After 120 min the mobile phase was changed to 100% methanol. After the final fractions were collected the column was washed for a further 1 hr with 100% methanol at 70 mL/min. Thirty-nine (39) fractions were collected over 120 min. Analytical HPLC was performed on all the fractions to determine their purity and FAEE profile, that closely matched those of the A fractions, and on that basis the following fractions were combined: A1 fr 25-30 and A1 fr 36-37.

Another variation of the A HPLC approach was conducted with these modifications: After 107 minutes the mobile phase was changed to 90% methanol/water. After 119 min the mobile phase was changed to 94% methanol/water. After 130 min the mobile phase was changed to 100% methanol. After the final fractions were collected the column was washed for a further 1 hr with 100% methanol at 70 mL/min. Forty (40) fractions were collected over 129 min. This provided isolation of high-purity DPAEE, and the fractions were designated A2 fr 25-30 and A2 fr 38-39.

Thereafter, fractions from method A were subjected to further purification steps and analyses. To obtain mid-purity DTA3EE, fractions A fr 39-40, A1 fr 36-37, and A2 fr 38-39 were combined and extracted with pet. spirit (3×300 mL). The combined pet. spirit layers were dried over anhydrous magnesium sulfate, filtered, and concentrated in vacuo. This preparation was designated AP (yield: 260 mg).

Preparative HPLC method B: For isolation of mid-purity DPAEE by preparative HPLC, the double-distilled FAEE oil was chromatographed under standard conditions and a Deltaprep C18 column, with modifications. The objective was to collect a single DPA fraction containing between 50-85% DPA. From the starting material, 1.07 g double-distilled DPAEE, a single fraction was collected from 72 min (15 min prior to the DPA peak) to 120 min (15 min after the end of the DPA peak) under these conditions: After 105 min the mobile phase was changed to 90% methanol/water. After 120 min, the mobile phase was changed to 94% methanol/water. After 124 min, the mobile phase was changed to 100% methanol. After the final fractions were collected, the column was washed for a further 1 hr with 100% methanol at 70 mL/min. The single fraction (designated B fr 1) was evaporated for GC analysis (yield: 338 mg).

In a related approach, HPLC method B was modified as follows: After 96 min the mobile phase was changed to 90% methanol/water. After 111 min the mobile phase was changed to 94% methanol/water. After 116 min the mobile phase was changed to 100% methanol. After the final peaks had eluted from the column, the column was washed for a further 1 hr with 100% methanol at 70 mL/min. Analytical HPLC was performed on the fraction, designated B1 fr1, to determine its purity and FAEE profile, which closely matched that of B fr 1.

In another related approach, HPLC method B was modified as follows: After 104 min the mobile phase was changed to 90% methanol/water. After 119 min the mobile phase was changed to 94% methanol/water. After 126 min the mobile phase was changed to 100% methanol. After the final peaks had eluted from the column, the column was washed for a further 1 hr with 100% methanol at 70 mL/min. A single fraction was collected from 15 min prior to the DPA peak to 15 min after the end of the DPA peak. Analytical HPLC was performed on the fraction, designated B2 fr1, to determine its purity and FAEE profile, which closely matched that of fraction B fr 1. This procedure was repeated to prepare fractions B1 fr 1 and B2 fr 1.

Thereafter, B fr 1, B1 fr 1, and B2 fr 1 were combined and extracted with pet. spirit (3×3 L). The combined pet. spirit layers were dried over anhydrous magnesium sulfate, filtered and concentrated in vacuo. This preparation was designated BL (yield: 1.1 g).

The concentrated DPAEE extract, BL, was chromatographed under the standard conditions using 94% methanol/water mobile phase. A total of eight fractions were collected over 24 min. Analytical HPLC was performed on all the fractions to determine their purity and FAEE profile, which closely matched those of BL, and on that basis the following fractions were combined and designated BM fr 1-8.

Subsequently, BM fr 1-8 were combined and extracted with pet. spirit (3×300 mL). The combined pet. spirit layers were dried over anhydrous magnesium sulfate, filtered and concentrated in vacuo. This DPAEE extract was designated BN (yield: 809 mg).

Preparative HPLC method C: An alternative isolation of high-purity DPAEE was conducted as follows. Crude DPA-juncea derived FAEE double-distilled oil (1.57 g) was chromatographed under standard conditions using a 250×50 mm Gemini-NX C18 column, with the following modifications: Fractions were collected from the beginning of the EPAEE peak at about 58 min. A total of twenty-one (21) fractions were collected over 73 min. After 111 min, the mobile phase was changed to 90% methanol/water. After 127 min the mobile phase was changed to 94% methanol/water. After 137 min the mobile phase was changed to 100% methanol. After the final fractions were collected the column was washed for a further 1 hr with 100% methanol at 70 mL/min. Analytical HPLC was performed on all the fractions and on that basis the following fractions were combined and concentrated in vacuo for GC analysis. Yields are shown in Table 3:

TABLE 3
Yield from preparative HPLC method C

| | Mass | Major |
|---|---|---|
| Fraction | obtained | component(s)* |
| C fr 1 | 10 mg | EPA |
| C fr 2-3 | 80 mg | ALA |
| C fr 5-6 | 50 mg | ETA |
| C fr 7-9 | 40 mg | LA, ETA |
| C fr 14-15 | 14 mg | ETrA, DPA3 |
| C fr 16-18 | 203 mg | OA |
| C fr 19-20 | 60 mg | DTA3 |
| C fr 10-13 | 283 mg | DPA3 |
| C fr 10-13aq | 6 mg | DPA3 |

*At least 10% FAEE.

Fractions C fr 10-13 were extracted with pet. spirit (3×300 mL). The combined pet. spirit layers were dried over anhydrous magnesium sulphate, filtered and concentrated in vacuo. The residual aqueous layer was then also concentrated in vacuo in order to determine the completeness of the extraction procedure. This step resulted in C fr 10-13 and C fr 10-13 aq with yields shown in Table 3, above.

A variation of method C was carried out with the following modifications: After 93 min the mobile phase was changed to 90% methanol/water. After 116 min the mobile phase was changed to 94% methanol/water. After 124 min the mobile phase was changed to 100% methanol. After the final fractions were collected the column was washed for a further 1 hour with 100% methanol at 70 mL/min. A total of 23 fractions were collected over 68 min. Fractions were combined as follows: C1 fr 5-6, C1 fr 10-13, and C1 fr 20-21.

Another variation of method C was carried out with the following modifications: After 96 min the mobile phase was changed to 90% methanol/water. After 111 min the mobile phase was changed to 94% methanol/water. After 117 min the mobile phase was changed to 100% methanol. After the final fractions were collected the column was washed for a further 1 hour with 100% methanol at 70 ml/min. A total of 23 fractions were collected over 70 min. Fractions were combined as follows: C2 fr 5-6, C2 fr 10-13, and C2 fr 20-21.

Yet another variation of method C was carried out with the following modifications: After 99 minutes the mobile phase was changed to 90% methanol/water. After 115 min the mobile phase was changed to 94% methanol/water. After 126 min the mobile phase was changed to 100% methanol. After the final fractions were collected the column was washed for a further 1 hr with 100% methanol at 70 ml/min. A total of 24 fractions were collected over 75 min. Analytical HPLC was performed on all the fractions to determine their purity and FAEE profile, which closely matched those of C fr, and on that basis the following fractions were combined. Fractions were combined as follows: C3 fr 5-6, C3 fr 10-13, and C3 fr 20-22.

An additional variation of method C was carried out with the following modifications: After 92 min the mobile phase was changed to 90% methanol/water. After 109 min the mobile phase was changed to 94% methanol/water. After 116 min the mobile phase was changed to 100% methanol. After the final fractions were collected the column was washed for a further 1 hour with 100% methanol at 70 mL/min. A total of 24 fractions were collected over 72 min. Analytical HPLC was performed on all the fractions to determine their purity and FAEE profile, which closely matched those of C fr, and on that basis the following fractions were combined. Fractions were combined as follows: C4 fr 5-6, C4 fr 10-13, and C4 fr 20-21.

Following these procedures, fractions C fr 5-6, C1 fr 5-6, C2 fr 5-6, C3 fr 5-6, and C4 fr 5-6 were combined and extracted with pet. spirit (3×300 mL). The combined pet. spirit layers were dried over anhydrous magnesium sulfate, filtered, and concentrated in vacuo. This preparation comprised high ETAn-3EE, and is designated CR (yield: 217 mg).

Additionally, following the initial HPLC procedures, fractions C fr 19-20, C1 fr 20-21, C2 fr 20-21, C3 fr 20-22, and C4 fr 20-21 were combined and extracted with pet. spirit (3×300 mL). The combined pet. spirit layers were dried over anhydrous magnesium sulfate, filtered and concentrated in vacuo. This preparation comprised high DTAn-3EE, and is designated CS (yield: 254 mg).

Also, following the initial preparative HPLC procedures, fractions C fr 10-13, C1 fr 10-13, C2 fr 10-13, C3 fr 10-13, and C4 fr 10-13 were combined and extracted with pet. spirit (3×3 L). The combined pet. spirit layers were dried over anhydrous magnesium sulphate, filtered and concentrated in vacuo. These preparations provided high-purity DPAEE, designated CT1 (yield: 1.088 g) and CT2 (yield: 417 mg).

The concentrated DPAEE extract (417 mg, CT2) was chromatographed under the standard conditions using 94% methanol/water mobile phase, and four fractions were collected over 12 min. Analytical HPLC was performed on all the fractions to determine the purity and FAEE profile, which closely matched those of CT2, and on that basis the following fractions were combined and designated CX fr 2-4. Additionally, the concentrated DPAEE extracts (1.088 mg, CT1) was chromatographed under the standard conditions using 94% methanol/water mobile phase, and six fractions were collected over 16 min. Analytical HPLC was performed on all the fractions to determine the purity and FAEE profile, which closely matched those of CT1, and on that basis the following fractions were combined and designated CZ fr 3-6.

Thereafter, concentrated DPAEE preparations CX fr 2-4 and CZ fr 3-6 were combined and extracted with pet. spirit (3×300 mL). The combined pet. spirit layers were dried over anhydrous magnesium sulfate, filtered and concentrated in vacuo. This preparation was designated CXZ (yield: 1.0 g).

Preparative HPLC method D: An alternative isolation of mid-purity DPAEE was also used to collect a single DPA fraction containing between 50-85% DPA. Crude DPA-juncea derived FAEE double-distilled oil (1.57 g), was chromatographed under standard conditions using a 250×50 mm Gemini-NX C18 column, with the following modifications: A single fraction was collected from 15 min prior to the DPA peak, to 15 min after the end of the DPA peak. After 90 min the mobile phase was changed to 90% methanol/water. After 100 min the mobile phase was changed to 94% methanol/water. After 113 min the mobile phase was changed to 100% methanol. After the final peaks had eluted from the column, the column was washed for a further 1 hr with 100% methanol at 70 ml/min. The single fraction was extracted with pet. spirit (3×300 mL). The combined pet. spirit layers were dried over anhydrous magnesium sulphate, filtered and concentrated in vacuo for GC analysis. This fraction was identified as D fr 1 (yield: 587 mg)

The distilled fatty acid ethyl esters (FAEE) of the reference blend were similarly subjected to chromatographic separation under similar conditions. Analytical HPLC was performed on all the fractions, and the “symmetrical” fractions containing predominantly EPA were combined.

Example fatty acid content in DPA Juncea crude oil and at various enrichment steps is shown in the following table (oil was analyzed by GC-FID as is well-known in the art; FAEE identities established using Supelco 37 FAME standard mix transesterified to FAEE mix):

TABLE 4
Example fatty acid content per enrichment

| Sample | OA | LA | ALA | SDA | ETA | EPA | DTA3 | DPA3 | DHA | N3 tot | N9 tot |
|---|---|---|---|---|---|---|---|---|---|---|---|
| Crude DPA oil | 43.73 | 7.64 | 16.51 | 1.05 | 3.55 | 0.54 | 1.88 | 10.46 | — | 34.58 | 45.14 |
| Transesterified | 43.73 | 7.64 | 16.51 | 1.05 | 3.55 | 0.54 | 1.88 | 10.46 | — | 34.58 | 45.14 |
| 1st Distillate | 48.30 | 8.46 | 18.29 | 1.25 | 2.49 | 0.40 | 0.61 | 3.75 | — | 27.18 | 49.27 |
| 1st Retentate | 36.90 | 6.27 | 13.93 | 0.64 | 5.01 | 0.72 | 3.48 | 19.18 | — | 43.78 | 39.38 |
| 2nd Distillate | 43.43 | 7.37 | 16.55 | 0.97 | 4.57 | 0.68 | 1.79 | 10.44 | — | 35.76 | 45.31 |
| 2nd Retentate* | 22.03 | 3.66 | 8.61 | — | 6.13 | 0.91 | 6.93 | 35.80 | — | 59.53 | 25.87 |
| DPA Fraction | — | 2.45 | 0.10 | — | 0.20 | — | — | 95.31 | — | 95.73 | — |
| ETA Fraction | — | 2.73 | 1.78 | — | 92.90 | 0.07 | — | 0.43 | — | 95.18 | — |
| DTA Fraction | 43.34 | 0.08 | 0.10 | — | 0.09 | — | 48.99 | 0.98 | — | 50.27 | 43.34 |
| DPA Fraction | 9.88 | 6.70 | 0.34 | — | 11.01 | — | 0.53 | 65.97 | — | 80.01 | 9.88 |

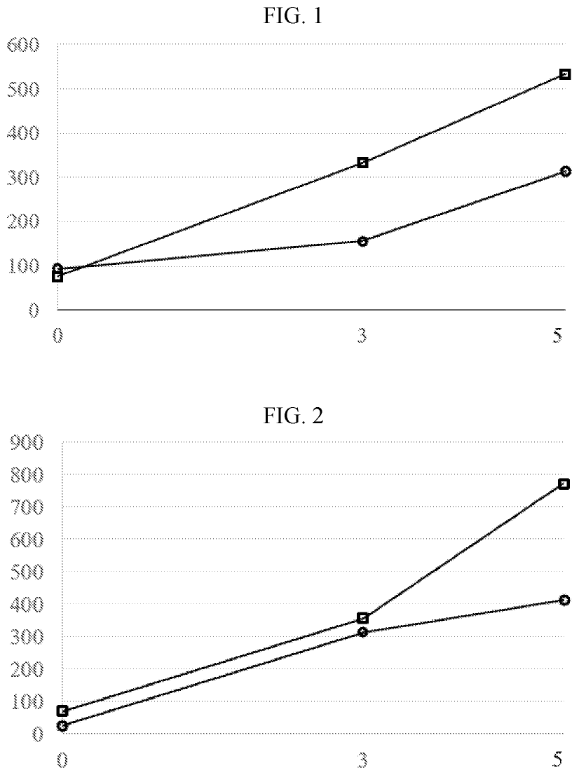

Crude oil = expeller press portion only, tot = total; Fractions via preparative HPLC of 2nd Retentate.
*This preparation used in stability comparison of FIG. 1.

A more detailed presentation of FA content of various enriched fractions of the present embodiments is shown in Table 5:

TABLE 5
GC analysis results for oils derived from DPA Juncea FAEE oil

| | | | | | | | | | Sum |
|---|---|---|---|---|---|---|---|---|---|
| Sample | OA | LA | ALA | ETrA | ETA | EPA | DTA3 | DPA3 | EPA, DPA |
| Transesterified | 43.73 | 7.64 | 16.51 | 0.59 | 3.55 | 0.54 | 1.88 | 10.46 | 11.0 |
| 2nd Retentate | 22.03 | 3.66 | 8.61 | 1.15 | 6.13 | 0.91 | 6.93 | 35.80 | 38.71 |
| A fr 18 | 0.08 | 0.06 | 33.87 | — | — | 60.24 | — | 0.07 | 60.32 |
| A fr 19-21 | — | — | 89.94 | — | 0.08 | 4.60 | — | — | 4.60 |
| A fr 23-26 | — | 2.73 | 1.78 | — | 92.90 | 0.07 | — | 0.43 | 0.51 |
| A fr 27-28 | — | 79.92 | 0.56 | — | 3.07 | — | — | 15.23 | 15.23 |
| A fr 29-34 | — | 2.45 | 0.10 | 0.12 | 0.20 | — | — | 95.31 | 95.31 |
| A fr 35-37 | 25.64 | 1.0 | 0.53 | 37.57 | 0.76 | — | 0.92 | 16.07 | 16.07 |
| A fr 38 | 87.36 | — | — | 0.11 | 0.0 | — | 3.77 | 0.52 | 0.52 |
| A fr 39-40 | 43.34 | 0.08 | 0.10 | 0.11 | 0.09 | — | 48.99 | 0.98 | 0.98 |
| AP | 55.79 | — | — | — | — | — | 37.36 | 0.60 | 0.60 |
| B fr 1 | 9.88 | 6.70 | 0.34 | 2.16 | 11.01 | — | 0.53 | 65.97 | 65.97 |
| BL | 11.83 | 6.82 | — | 1.97 | 10.86 | — | 0.85 | 63.33 | 63.33 |
| BN | 11.82 | 6.96 | 0.40 | 2.11 | 11.01 | — | 0.25 | 64.31 | 64.31 |
| C fr 1 | 0.17 | — | — | — | — | 93.04 | — | — | 93.04 |
| C fr 2-3 | 0.11 | — | 89.91 | — | — | 4.10 | — | — | 4.10 |
| C fr 5-6 | — | — | 0.54 | — | 94.89 | 0.06 | — | 0.18 | 0.26 |
| C fr 7-9 | 0.01 | 80.81 | 0.30 | — | 14.92 | 0.02 | — | 0.38 | 0.43 |
| C fr 10-13 | — | 0.18 | — | 0.23 | 0.06 | — | — | 98.42 | 98.42 |
| C fr 10-13 aq | 0.03 | 0.18 | — | 0.18 | 0.07 | 0.03 | — | 98.11 | 98.11 |
| C fr 14-15 | 0.14 | 0.17 | 0.06 | 54.06 | 0.16 | — | 0.40 | 34.58 | 34.58 |
| C fr16-18 | 91.25 | 0.04 | — | — | — | — | 1.34 | 0.40 | 0.40 |
| C fr 19-20 | 6.30 | 0.02 | — | 0.03 | — | — | 86.59 | 0.32 | 0.32 |
| CR | 0.05 | — | 0.71 | — | 98.45 | 0.05 | — | — | 0.05 |
| CS | 11.53 | — | 0.44 | 0.03 | — | — | 80.57 | — | 0.35 |
| CT | 0.13 | 0.29 | — | 0.12 | 0.09 | — | — | 96.68 | 96.68 |
| CXZ | 0.10 | 0.31 | — | 0.13 | — | — | — | 98.61 | 98.61 |
| D fr 1 | 30.14 | 4.99 | 0.07 | — | 8.22 | — | 3.36 | 48.11 | 48.11 |

In some embodiments, fractions may be blended to achieve a desired concentration of particular FAEE. For example, enriched DPA fractions may be blended with another fraction, or with an oil from a different source (e.g., DHA Canola oil or another canola oil), to provide a lipid composition comprising about 45% DPAn-3. In at least one embodiment, the composition comprises 20%-50% DPAn-3, 10%-30% OA, and 2%-20% ETA (all ranges inclusive).

### Example 6—Oil Stability Tests

A selection of oils was subjected to light-induced, time course, accelerated oxidative studies using solid phase micro-extraction (SPME) head space GC/MS. This was to determine if the plant-derived oils showed greater stability than the marine-derived reference blend oils, which was indeed observed.

Headspace GC-MS stability trial was done as follows. Headspace analysis was conducted on the enriched products prepared as described above to assess the quantities of propanal that are released under specific conditions. Increased levels of propanal release demonstrate reduced stability of the test material.

SPME (solid-phase microextraction) method: Selected 65 μm PDMS/DVB StableFlex fiber (Supelco fiber kit 57284-u). Fibers were conditioned for 10 min prior to use at 250° C. in a Triplus RSH conditioning station. Samples were incubated at 40° C. for 1 min prior to extraction.

GC method: Thermo Scientific TRACE 1310 GC Thermo Scientific TR-DIOXIN 5MS column, 0.25 mm internal diameter, 30 m film 0.1 μm. Split injection 250° C. Split 83, 1.2 ml He/min. GC Ramp: 40° C. 1 min to 100 at 5° C./min, then to 300° C. at 50° C./min.

A generic MS specific column with good synergy for headspace analysis was used. A slow initial temperature ramp was employed to maximize separation of volatiles before ramping up to maximum to maintain column performance. Split injections were employed to avoid the requirement for cryogenic cooling of the inlet and enhance column resolution.

Thermo Scientific DFS High resolution double focusing MS with TRACE 1310 and Triplus RSH auto-sampler employing High Resolution Multiple Ion Detection (MID) (LINEAR ELECTRIC SCAN) @10,000 resolution monitoring the following ions: m/z 57 Propanal —H (—H gives a higher dynamic range 58 was recorded but not used). Perflurokerosene (PFK) was employed as calibration and lock mass standard m/z 51, 69 and 93.

MS method: Thermo Scientific DFS high 5 resolution GC-MS, Low resolution (1000), full scan 35-350 Da at 0.5 s/scan, Standards: Propanal and Hexanal standard dilutions were made into supplied commercial canola oil. These Standard mixtures were then added at a volume of 100 μl to 20 ml headspace vials.

Full scan was employed, allowing the monitoring of all evolved products rather than specific molecules.

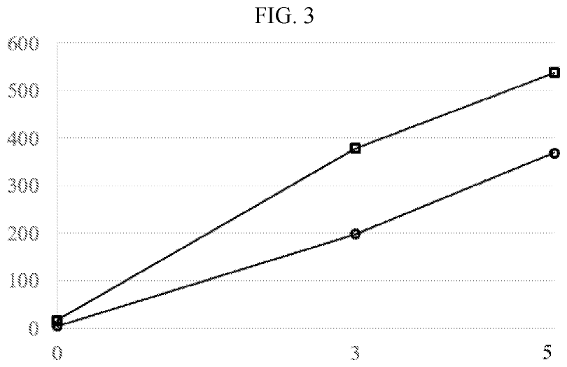

Stability Results: Table 6, below, provides results obtained from the DPA Juncea oil compared with reference preparations, obtained from the HPLC enrichment of Example 5 at T=0, T=3, and T=5 days. Test samples were held during this period at ambient temp on a light box and under fluorescent tube lighting. The m/z 57 molecular ion was analyzed, and the mass chromatogram clearly shows emergence of propanal at RT 1.37 mins. Propanal development is quantified in the table below, and the data are also illustrated in FIG. 1 to FIG. 3. The DPA Juncea oil released substantially lower amounts of propanal, thus demonstrating the improved stability of the FAEE fractions as compared with the reference compositions.

TABLE 6
Data from stability comparison

| Propanal (ppm) per | TO | T3 | T5 |
|---|---|---|---|
| time point (days) | (ppm) | (ppm) | (ppm) |
| DPA Juncea oil FAEE double distilled | 94 | 156 | 314 |
| Reference blend oil FAEE double distilled | 76 | 333 | 534 |
| DPA Juncea oil FAEE double distilled | 25 | 313 | 412 |
| and chromatographed, comprising | | | |
| 98% DPAn-3* | | | |
| Reference blend oil FAEE double distilled | 70 | 356 | 771 |
| and chromatographed, comprising | | | |
| 90% EPA | | | |
| DPA Juncea oil FAEE double distilled | 4 | 198 | 369 |
| and chromatographed, comprising | | | |
| 64% DPAn-3** | | | |
| Reference blend oil FAEE double distilled | 16 | 378 | 538 |
| and chromatographed, comprising | | | |
| 58% EPA | | | |

*Sample CXZ, FA content comprised 98.61% DPA and <1% any other single FA.
**Sample BN, FA content comprised 1.09% palmitic acid (16:0), 11.82% OA, 2.11% ETrA (20:3n3), 6.96% LA, 11.01% ETA, 64.31% DPAn-3, and <1% any other single FA.

These data show that FAEE prepared from DPA Juncea oil have superior stability compared with FAEE of the reference blend oil.

### Example 7—Modulation of Inflammatory Cytokine Production

The DPA, DTA, and ETA lipid compositions of the present embodiments modulate the immune system. The immune system is an organized complex network of biological structures and processes that protect against infectious disease. For example, cytokines and chemokines mediate interactions between cells directly, regulating target immune cell responses and promoting inflammation. These responses result in a coordinated attack where the immune system attempts to eradicate foreign pathogens and begin the healing process. Consequently, the inflammatory process plays a key protective role in immunity. In addition cytokine and chemokine research is essential in understanding the immune system and its multi-faceted response to most antigens, as well as disease states such as autoimmune disease, allergic reactions, sepsis and cancer. As beneficial as the immune response can be for protection against pathogens, excessive or inappropriate immunity can be harmful. For instance, it is proposed that chronic inflammation can contribute to diseases as diverse as type 2 diabetes, metabolic syndrome, liver disease, arthritis, atherosclerosis, cancer, colitis and neurodegenerative diseases. Therefore, the suppression of immunity can also be beneficial.

This Example investigates DPA-Juncea derived lipid compositions to confirm their immuno-modulatory activity and compare that activity to synthetic counterparts. The comparison, in at least one embodiment, shows that the vegetable-derived lipid compositions described herein are distinguishable from synthetic counterparts.

Preparation of test materials: Example lipid compositions for comparison in this Example include enriched FAEE compositions, as described herein, blended with synthetic fatty acids and reference oil fractions for comparison.

Free fatty acids were prepared from the FAEE of the previous examples: BrJDD (DPA Juncea FAEE double-distilled), BN, CS, and AP. To a 100 ml two-necked round-bottomed flask was added pet. spirit (10 ml) and fatty acid ethyl ester (150 mg) to form a clear solution. Lipozyme 435 (150 mg) was added and then deionized water (5 ml) and the mixture stirred vigorously with the flask immersed in an oil bath maintained at 40° C. The reaction mixture was checked daily by 1H NMR and TLC until the majority of the FAEE was seen to be diminished and no further hydrolysis was observed. Once completed the turbid white reaction mixture was allowed to cool to room temperature. The mixture was diluted with 100 ml fresh pet. spirit and transferred to a separatory funnel. The clear aqueous phase was removed, and the organic phase was filtered under vacuum to remove all solids. The clear filtrate was dried over anhydrous magnesium sulfate, filtered and concentrated in vacuo to give a viscous oil. Samples were analyzed by ¹H NMR (CDCl₃) and purified by radial chromatography (4 mm silica, eluting with 100% pet. spirit to 100% DCM, then 95:5 DCM: MeOH). Fractions were analyzed via TLC developed using 75:25 pet. spirit:EtOAc. The developed plates were sprayed with a basic bromocresol spray and fractions showing bright yellow spots were analyzed by 41 NMR. The fractions which contained free fatty acids were combined, concentrated in vacuo, and placed in vials and sealed under nitrogen.

Free fatty acids were also prepared from TAG oils from flax seed (Flx) and high oleic acid sunflower (HOS) as follows: To a 250 ml two-necked round-bottomed flask was added pet. spirit (60 ml) and triglyceride oil (1000 mg) to form a clear solution. Lipozyme 435 (1000 mg) was added and then deionized water (40 ml) and the mixture stirred vigorously with the flask immersed in an oil bath maintained at 40° C. The reaction mixture was checked daily by ¹H NMR and TLC until the majority of the triglyceride signals at 5.2, 4.2 and 4.1 ppm were seen to be diminished and no further hydrolysis was observed. Once completed the turbid white reaction mixture was allowed to cool to room temperature. The mixture was diluted with 100 ml fresh pet. spirit and transferred to a separatory funnel. The clear aqueous phase was removed, and the organic phase was filtered under vacuum to remove all solids. The clear filtrate was dried over anhydrous magnesium sulphate, filtered and concentrated in vacuo to give a viscous oil. The crude oil was analyzed by ¹H NMR (CDCl₃) and purified by radial chromatography (4 mm silica, eluting with 100% pet. spirit to 100% DCM, then 95:5 DCM: MeOH). Fractions were then analyzed via TLC developed using 75:25 pet. spirit:EtOAc. The developed plates were sprayed with a basic bromocresol spray and fractions showing bright yellow spots were analyzed by ¹H NMR. The fractions which contained free fatty acids were combined, concentrated in vacuo, and placed in vials and sealed under nitrogen.

Additionally, DPA (synDPA) and ETA (synETA) were purchased for use as comparators in the cell assays.

Various blends of free fatty acids were prepared as follows:

TABLE 7
Free fatty acid (FFA) blends for cell inflammatory marker assays

| | FFA Component | | | | | | | | |
|---|---|---|---|---|---|---|---|---|---|
| Blend Code | BrJdd | BN | CS | AP | Flx | HOS | | SynDPA | SynETA |
| BrJddFlxHOS | 96 mg | | | | 13 mg | 27 | mg | | |
| BrJddFlxHOS1 | 43 mg | | | | 15 mg | 44 | mg | | |
| Bfr1FlxHOS | | 102 mg | | | 23 mg | 28 | mg | | |
| Afr39-40HOS | | | | 14 mg | | 14 | mg | | |
| Cfr19-20HOS | | | 18 mg | | | 6 | mg | | |
| SynDHOS | | | | | | 33.5 | mg | 68 mg | |
| SynDEHOS | | | | | | 33 | mg | 80 mg | 6 mg |
| SynDEHOS1 | | | | | | 23 | mg | 74 mg | 13.5 mg |
| SynDEHOS2 | | | | | | 15 | mg | 61 mg | 14.4 mg |

The FA content of the FFA components and FFA blends was determined by GC and is shown in Table 8:

TABLE 8
GC analysis results for FFA and FFA blends (as % or total FA content of sample)

| | | | | | | | | | | Sum EPA, |
|---|---|---|---|---|---|---|---|---|---|---|
| Sample | OA | LA | ALA | ETrA | ETA | EPA | DTA3 | DPA3 | DHA | DPA, DHA |
| BrJ | 41.9 | 7.2 | 14.4 | 0.7 | 3.1 | 0.6 | 1.8 | 9.0 | — | 9.5 |
| BrJdd | 29.2 | 5.9 | 13.5 | 0.9 | 5.2 | 0.8 | 4.9 | 28.4 | — | 29.2 |
| BrJddFlxHOS | 36.2 | 8.3 | 16.5 | 0.6 | 3.5 | 0.6 | 3.4 | 19.7 | | 20.4 |
| BrJddFlxHOS1 | 46.2 | 10.6 | 16.2 | 0.4 | 2.1 | 0.4 | 1.9 | 10.8 | 0.1 | 11.2 |
| Bfr1FlxHOS | 23.9 | 9.5 | 13.6 | 1.4 | 6.6 | 0.5 | 0.9 | 38.1 | — | 38.6 |
| BN | 12.2 | 7.2 | 0.4 | 2.2 | 10.8 | — | 0.8 | 61.7 | 0.3 | 62.0 |
| SynDHOS | 23.4 | 4.7 | — | — | — | — | 0.4 | 67.4 | 0.2 | 67.5 |
| SynDEHOS | 19.8 | 3.4 | — | — | 5.7 | — | 0.4 | 66.8 | 0.2 | 67.0 |
| SynDEHOS1 | 12.0 | 2.2 | — | — | 10.3 | — | 0.4 | 72.5 | 0.2 | 72.7 |
| SynDEHOS2 | 13.8 | 2.3 | — | — | 17.3 | — | 0.4 | 63.3 | 0.1 | 63.4 |
| CXZ | 0.9 | 0.4 | — | 0.1 | — | — | — | 95.8 | — | 95.8 |
| SynDPA | — | — | — | — | — | — | — | 98.9 | 0.2 | 99.1 |
| AP | 59.5 | — | 0.1 | 0.1 | 3.0 | — | 29.9 | 0.1 | — | 0.1 |
| CS | 13.2 | — | 0.4 | — | 3.7 | — | 74.4 | 0.4 | — | 0.4 |
| HOS | 74.4 | 13.9 | 0.2 | — | — | — | — | — | — | — |
| Afr39-40HOS | 73.0 | 9.2 | — | — | 0.9 | — | 7.4 | 0.2 | — | 0.2 |
| Afr19-20HOS | 34.8 | 4.6 | 0.4 | — | — | — | 50.0 | 0.3 | 0.3 | 0.6 |
| CR | 0.6 | 0.1 | 0.7 | — | 96.2 | 0.1 | — | 0.1 | — | 0.2 |
| SynETA | — | — | — | — | 99.0 | — | — | — | — | — |
| Rfdd | 20.8 | 6.4 | 13.0 | 0.1 | 0.9 | 17.3 | — | 3.2 | 10.6 | 31.1 |
| DHAfr21-25* | 9.8 | 3.2 | 2.2 | — | 10.7 | — | — | — | 66.5 | 66.5 |

*Prepared from seed oil of canola line NSB500274 via HPLC method A.

Modulation testing: Briefly, spleen cells (splenocytes) are obtained from female mice (BALB/c mic) and cultured in 96-well plates. The cells are exposed to each oil preparation (in dilution) and lipopolysaccharide (LPS, typically from or otherwise mimicking the bacterium E. coli) to stimulate the cytokine response in the presence of the test oil preparation. Subsequently (after 24 hr culture with exposure to the oil preparation dilution and LPS), the media from the stimulated cells are tested for the presence of cytokines (i.e., cytokines that have been released from the cell into the media). Standard kits or sets of cytokines and antibodies useful for identification or quantification of cytokine modulation are available commercially. For example, the Milliplex Map Mouse Cytokine/Chemokine Magnetic Bead Panel (Millipore #MCYTMAG-70K-PX32) includes a premix of antibodies that recognize the following cytokine/chemokine analytes: Eotaxin/CCL11, G-CSF, GM-CSF, IFN-γ, IL-la, IL-10, IL-2, IL-3, IL-4, IL-5, IL-6, IL-7, IL-9, IL-10, IL-12 (p40), IL-12 (p′70), IL-13, IL-15, IL-17, IP-10, KC, LIF, LIX, MCP-1, M-CSF, MIG, MIP-1α, MIP-1β, MIP-2, RANTES, TNF-α, and VEGF. Data are normalized for cytokines to unstimulated controls.

Data may indicate that vegetable-derived DPA-enriched lipid compositions possess unexpected modulatory activity compared with synthetic lipid compositions.

### Example 8—Inhibition of Inflammatory Cytokines

A preliminary in vitro study of the effect of various free fatty acid (FFA) preparations on human blood cells activated by endotoxin (LPS) was undertaken. More specifically, the assay measured the effect in vitro of various FFA preparations on the ability of LPS-stimulated human peripheral blood mononuclear cells (PBMCs) to produce pro- and anti-inflammatory cytokines, chemokines, and growth factors.

Initially, three human PBMC samples were plated in 96-well plates and stimulated with four different LPS doses against an LPS-free control (i.e., 0, 1, 10, 100, 1000 ng/mL). Each series was also incubated with a single FFA preparation, Rfdd (see Table 8), at three different doses (against a FFA-free control) in a DMSO vehicle. The cellular distributions of the samples were determined based on flow cytometric analysis of cell surface markers (CD3, CD4, CD8, CD14, CD19, CD56). Table 9 provides further information on the source and cellular content of the human cell samples:

TABLE 9
Human PBMC samples

| | | | | B | | NK |
|---|---|---|---|---|---|---|
| Donor | Age | Sex | T cells (CD4, CD8) | cells | Monocytes | cells |
| D1 | 29 y | M | 38% (48, 32) | 14% | 20% | 17% |
| D2 | 56 y | F | 37% (52, 43) | 13% | 37% | 8% |
| D3 | 49 y | M | 39% (56, 39) | 20% | 27% | 13% |

The production of thirty-eight different cytokine/chemokine/growth factors (MILLIPLEX map human Cytokine/Chemokine Magnetic Bead Panel—Premixed 38 Plex-Immunology Mulitplex Assay (Millipore HCYTA-60K-PX38)) was compared among these cell samples. The markers in this assay kit were EGF, Eotaxin/CCL11, G-CSF, GM-CSF, IFNα2, IFNγ, IL-1α, IL-1β, IL-RA, IL-2, IL-3, IL-4, IL-5, IL-6, IL-7, IL-8, IL-10, IL-12 (p40), IL-12 (p′70), IL-13, IL-15, IL-17A, IL-17E/IL-25, IL-17F, IL-18, IL-22, IP-10, MCP-1, M-CSF, MIG, MIP-1α, MIP-1β, PDGF-AA, PDGF-AB/BB, RANTES, TNFα, TNFβ, and VEGF-A. Data showed that 1 ng/mL LPS exhibited robust stimulatory effects on levels of multiple analytes.

In the absence of FFA preparation, LPS activated monocytes generally as expected. Specifically, the following factors were induced by LPS: Eotaxin (slightly), G-CSF, GM-CSF, IFNγ, IL-la and IL-10, IL-1RA, IL-6, IL-8 (slightly), IL-10, IL-12 (p40) (2/3 samples), IL-17e, IL-18, IL-22, MIP1α, MIP1β, RANTES (slight), TNFα, and TNFβ. In contrast, three factors were suppressed: IL-2, IP10, MCP-1 (2/3samples), although it is not clear why these three factors would be suppressed by LPS. Note that T cell derived cytokines in general were not activated; B cells may also have contributed to LPS-induced expression. When stimulated with 1 ng/mL of LPS, Rfdd exhibited moderate inhibitory effects on D1 PBMCs as shown in decreases in cytokines such as IFNγ, IL-1β, IL-1RA, IL-12, and TNFα. PBMC from D1 was selected for further study.

D1 PBMC were thawed and plated in 96-well plates at a density of 1×10⁵ cells per well. The plated cells were then treated with blinded test FFA preparations (see Example 7) serially diluted at 1:3 dilutions with a highest dose at approximately 30 in duplicates. For context, the typical total FFA content in blood of −580 μM for normal people after overnight fasting. Treated cells were then stimulated with 1 ng/mL of LPS. Control wells were set up with untreated PBMCs that were either stimulated or unstimulated with 1 ng/mL of LPS in the presence of vehicle (0.1 or 0.3% DMSO). Cell-free supernatant was collected 24 hours post treatment and analyzed with the Human 38 plex cytokine/chemokine/growth factor panel A Milliplex Map Kit (Millipore HCYTA-60KPX38) noted above.

Regarding inhibition, an inhibitory dose was considered any preparation that generated a signal less than 50% that of the control (LPS-stimulated, no added FFA) signal. To analyze the data in the context of FFA content, a simple scale from 0-5 for inhibition based primarily on the 30 μM data was created based on the following qualitative aspects: 0 was used when there was no or very minimal inhibition. IFNγ inhibition as well as inhibition of the cytokines IL-1 series (IL-1α, IL-1β, IL-1RA) and the chemokines IP-10 and MCP-1 were considered low and ranked from 1 to 2.5. A score of 3 was given if TNFα was also inhibited. Above 3 required inhibition of some T cell cytokines. The maximum of 5 was assigned if all cytokines were inhibited. Results are shown in Table 10 (FA content rounded, 0 means <0.5):

TABLE 10
In vitro inhibition LPS-stimulated cytokines by 30 μM FFA preparations

| | | | | | | | | | | | Inhibitory |
|---|---|---|---|---|---|---|---|---|---|---|---|
| | | | | | | | | | | Inhibited | scale |
| Prep | OA | LA | ALA | ETrA | ETA | EPA | DTA3 | DPA3 | DHA | Targets | (0-5) |
| Rfdd | 21 | 6 | 13 | 0 | 1 | 17 | — | 3 | 11 | IFNγ, IL-1 series, | 2 |
| | | | | | | | | | | IP-10/MCP-1 | |
| BrJdd | 29 | 6 | 14 | 1 | 5 | 1 | 5 | 28 | | IFNγ, IL-1 series, | 3 |
| | | | | | | | | | | IP-10/MCP-1, | |
| | | | | | | | | | | and TNF | |
| BN | 12 | 7 | 0 | 2 | 11 | — | 1 | 62 | 0 | nil | 0 |
| CXZ | 1 | 0 | — | 0 | — | — | — | 96 | — | IFNγ, IL-1 series, | 4.5 |
| | | | | | | | | | | IP-10/MCP-1, | |
| | | | | | | | | | | TNFα, T cell | |
| | | | | | | | | | | cytokines | |
| CR | 1 | 0 | 1 | — | 96 | 0 | 0 | 0 | — | IFNγ, IL-1 series, | 2 |
| | | | | | | | | | | IP-10 | |
| CS | 14 | — | 0 | — | 4 | — | 74 | 0 | — | IFNγ, IL-1 series, | 3.5 |
| | | | | | | | | | | IP-10/MCP-1, | |
| | | | | | | | | | | TNF | |
| AP | 60 | — | 0 | 0 | 3 | — | 30 | 0 | — | nil | 0 |
| DHA fr | 10 | 3 | 2 | — | 11 | — | — | — | 66 | nil | 0 |
| 21-25 | | | | | | | | | | | |
| BrJ | 42 | 7 | 14 | 1 | 3 | 1 | 2 | 9 | — | IFNγ, IL-1 series, | 3.75 |
| | | | | | | | | | | IP-10/MCP-1, | |
| | | | | | | | | | | some T cell | |
| | | | | | | | | | | cytokines, not | |
| | | | | | | | | | | TNF | |
| HOS | 74 | 14 | 0 | — | — | — | — | — | — | IFNγ, IL-1 series, | 4.0 |
| | | | | | | | | | | IP-10/MCP-1, | |
| | | | | | | | | | | TNFα, some T | |
| | | | | | | | | | | cell cytokines | |
| BrJdd | 36 | 8 | 16 | 1 | 4 | 1 | 3 | 20 | — | IFNγ, partial | 1.5 |
| Flx HOS | | | | | | | | | | IL-1 series, IP10 | |
| BrJdd | 46 | 11 | 16 | 0 | 2 | 0 | 2 | 11 | 0 | IFNγ, IL-1 series, | 2 |
| Flx HOS1 | | | | | | | | | | IP-10/MCP-1 | |
| B fr1 | 24 | 10 | 14 | 1 | 7 | 1 | 1 | 38 | — | IFNγ, IL-1 series, | 2.25 |
| Flx HOS | | | | | | | | | | IP-10/MCP-1 | |
| A fr | 73 | 9 | 0 | — | 1 | — | 7 | 0 | — | IFNγ, partial IL- | 1.5 |
| 39-40 HOS | | | | | | | | | | 1 series, IP-10 | |
| A fr | 35 | 5 | 0 | — | — | — | 50 | 0 | 0 | IFNγ, partial IL- | 1.5 |
| 19-20 HOS | | | | | | | | | | 1 series, MIP1a | |
| Syn | — | — | — | — | — | — | — | 99 | 0 | IFNγ, IL-1 series, | 4.0 |
| DPA | | | | | | | | | | IP-10/MCP-1, | |
| | | | | | | | | | | TNFα, some T | |
| | | | | | | | | | | cell cytokines | |
| SynD | 23 | 5 | — | — | — | — | 0 | 67 | 0 | IFNγ, IL-1 series, | 4.0 |
| HOS | | | | | | | | | | IP-10/MCP-1, | |
| | | | | | | | | | | TNFα, some T | |
| | | | | | | | | | | cell cytokines | |
| Syn | — | — | — | — | 99 | — | — | — | — | IFNγ, IL-1 series, | 4.75 |
| ETA | | | | | | | | | | IP-10/MCP-1, | |
| | | | | | | | | | | TNFα, T cell | |
| | | | | | | | | | | cytokines | |
| Syn DE | 20 | 3 | — | — | 6 | — | 0 | 67 | 0 | IFNγ, IL-1 series, | 3.8 |
| HOS | | | | | | | | | | IP-10/MCP-1, | |
| | | | | | | | | | | some T cell | |
| | | | | | | | | | | cytokines, some | |
| | | | | | | | | | | TNF | |
| Syn DE | 12 | 2 | — | — | 10 | — | 0 | 72 | 0 | IFNγ, IL-1 series, | 3.5 |
| HOS1 | | | | | | | | | | IP-10/MCP-1, | |
| | | | | | | | | | | some T cell | |
| | | | | | | | | | | cytokines, not | |
| | | | | | | | | | | TNF | |
| Syn DE | 14 | 2 | — | — | 17 | — | 0 | 63 | 0 | IFNγ, IL-1 series, | 5.0 |
| HOS2 | | | | | | | | | | IP-10/MCP-1, | |
| | | | | | | | | | | TNFα, T cell | |
| | | | | | | | | | | cytokines | |

Generally speaking, a few FFA preparations displayed strong inhibitory effect at the 30 μM dose (>50% reduction compared to LPS-stimulated control) on more than half the analytes: combined high B. juncea DPAn-3 fractions (˜95.8% DPAn-3); synthetic ETA; and SynDEHOS2 (synthetic DPA and EPA with oleic acid).

Some of the compounds had strong inhibitory effects. IFNγ was universally inhibited by all FFA preparations, at least weakly. Other types of minimal inhibition tended to include cytokines IL-1α, IL-1β, and IL-1RA (IL-1 series) or the chemokines IP-10 and MIP-1. The most potent of inhibitory formulations also suppressed classic T cell cytokines such as IL2-7, IL-13, IL-15, and IL-22. The most inhibitory preparations also inhibited TNFα, which could be considered a representative marker of strong inhibition. In this preliminary study, it appeared that at least one DPA preparation derived from DPA Juncea (˜96% DPAn-3 from the C series of techniques described herein) was more inhibitory than the analogous synthetic DPA. Overall, preparations containing a higher amount of DPAn-3, DTAn-3, or ETA had inhibitory activity in this assay. ETA, DTAn-3, and DPAn-3 all displayed significant inhibitory activity when present as the primary component in the preparation. Surprisingly, combinations of DPAn-3 and ETA were effective inhibitors of inflammatory cytokines in this assay.

Immunomodulatory activity was also observed for FFA preparations provided at approximately 10 μM. More specifically, a stimulatory signal was categorized as a signal above 130% compared with LPS-stimulated control (no FFA added), and an inhibitory signal was categorized as a signal below 70% compared with LPS-stimulated (no FFA added). At 10 μM, the following preparations displayed stimulatory responses only (see Table 8 for FFA content): BrJdd, BN, Bfr1F1×HOS, and Rfdd. At 10 μM, the following preparations displayed inhibitory responses: CR (IFNγ, IL-12(p40), IP-10 only), AP (IL-12(p40), IL-17F only), DHAfr21-25 (IL12(p40), IL-17F only), HOS (substantial), and SynDEHOS1 (six cytokines). At 10 μM, the following preparations displayed both stimulatory and inhibitory responses: CXZ, CS, BrJddFlxHOS, BrJddFlxHOS1, Afr39-40HOS, Afr19-20HOS, SynDPA, SynDHOS, SynETA, SynDEHOS, and SynDEHOS2.

Further regarding stimulation, in general, the only cytokine that was increased consistently with the 30 μM preparation was GM-CSF (in 15/21 formulations over 150% compared with control).

Although the preceding embodiments have been described in some detail by way of illustration and example for purposes of clarity and understanding, it will be clear to one of skill in the art that certain changes and modifications may be practiced within the scope of the invention which is limited solely by the appended claims.

The listing or discussion of an apparently prior-published document in this specification should not necessarily be taken as an acknowledgement that the document is part of the state of the art or is common general knowledge.

1. A lipid composition enriched for at least one of DPAn-3, DTAn-3, ETA, or ETrA, as described herein.

2. A lipid composition enriched for at least one of DPAn-3, DTAn-3, ETA, or ETrA, wherein at least one of DPAn-3, DTAn-3, ETA, or ETrA is enriched from a plant source.

3. The lipid composition of claim 2, wherein the DPAn-3, DTAn-3, ETA, or ETrA are ethyl esters or triglycerides.

4. The lipid composition of claim 2 or claim 3, wherein said composition exhibits improved stability compared with similar compositions derived from a marine source.

5. A lipid composition comprising an enriched seed oil-derived DPAn-3, wherein said composition comprises 20%-50% DPAn-3, 10%-30% OA, and 2% to 20% ETA.

6. The lipid composition of claim 5, comprising about 36% DPAn-3, about 22% OA, and about 6% ETA.

7. A lipid composition comprising an enriched seed oil-derived DPAn-3, wherein said composition comprises about 10.5% DPAn-3, about 44% OA, and about 4% ETA.

8. A lipid composition comprising an enriched seed oil-derived DPAn-3, wherein the composition comprises 60%-70% DPAn-3 and 0%-20% ETA.

9. A seed oil-derived enriched lipid composition comprising 90%-99% DPAn-3.

10. A lipid composition comprising an enriched seed oil-derived DTAn-3, wherein the composition comprises 40%-95% DTAn-3 and 5%-60% ETA.

11. A seed oil-derived enriched lipid composition comprising 90%-99% DTAn-3.

12. A seed oil-derived enriched lipid composition comprising 90%-99% ETA.

13. A lipid composition comprising an enriched seed oil-derived DPAn-3, wherein the composition comprises 10%-40% DPAn-3 and 20%-60% ETrA, and 0%-30% OA.

14. The composition of claim 13, comprising about 37% ETrA and about 16% DPAn-3.

15. The lipid composition of any one of the preceding claims, wherein the plant or seed is a Brassicaceae.

16. The lipid composition of claim 15, wherein the Brassicaceae is B. juncea or B. napus.

17. The lipid composition of claim 16, wherein the B. juncea is NUBJ1207, ATCC Accession No. PTA-125954.

18. A lipid composition that inhibits inflammatory cytokine production, comprising DPAn-3 and ETA.

19. The composition of claim 18, wherein the DPAn-3 and ETA are enriched from vegetable oil.

20. The composition of claim 18, comprising about 28% DPAn-3, about 5% DTAn-3, about 5% ETA, about 14% ALA, about 6% LA, and about 29% OA.

21. A lipid composition that inhibits inflammatory cytokine production, comprising about 74% DTAn-3, about 14% OA, and about 4% ETA.

22. A lipid composition that inhibits inflammatory cytokine production, comprising about 96% DPAn-3 and about 1% OA.

23. A lipid composition that inhibits inflammatory cytokine production, comprising about 96% DPAn-3 enriched from a seed oil of B. juncea is NUBJ1207, ATCC Accession No. PTA-125954.

24. The lipid composition of any one of the preceding claims, wherein the composition is provided in the form of a tablet, capsule, encapsulated gel, ingestible liquid or powder, or a topical ointment or cream.

25. A lipid composition as defined in any of claims 1 to 24 for use in the treatment or prevention of cardiovascular disease, protection against death in patients with cardiovascular disease, reduction of overall serum cholesterol levels, reduction in high blood pressure, increase in HDL:LDL ratio, reduction of triglycerides, or reduction of apolipoprotein-B levels.
